(19) United States
(12) Patent Application Publication
(10) Pub. No.: US 2005/0037485 A1
(43) Pub. Date: Feb. 17, 2005

(54) System and method for process automation

(76) Inventors: Seth T. Rodgers, Somerville, MA (US); Ian K. MacGregor, Merrimack, NH (US); Todd A. Basque, Danvers, MA (US); Scott E. Miller, Somerville, MA (US); Zhimin Lu, Chelmsford, MA (US); Andrey J. Zarur, Winchester, MA (US); Mohamed Shaheen, Methuen, MA (US)

Correspondence Address: Timothy J. Oyer, Ph.D.;Wolf, Greenfield & Sacks, P.C., 600 Atlantic Avenue, Boston, MA, 02210, US

(21) Appl. No.: 10/863,585

(22) Filed: Jun. 7, 2004

Related U.S. Application Data
(63) Continuation-in-part of application No. 10/457,017, filed on Jun. 5, 2003, now abandoned.

(51) Int. Cl. C12M 1/34; C12Q 1/70

(52) U.S. Cl. 435/287.2

(57) ABSTRACT

Disclosed are systems and methods for manipulating chemical, biological, and/or biochemical samples, optionally supported on substrates and/or within chambers, for example biological samples contained on chips, within biological chambers, etc. In certain embodiments, an apparatus configured to be able to position a chamber or other substrate in one or more modules surrounding the apparatus is disclosed. The apparatus may be configured to be able to move the chamber or substrate in any set of directions, such as radially, vertically, and/or rotationally, with respect to the apparatus. The apparatus may be manually operated and/or automatically controlled. Examples of modules include, but are not limited to, stacking or holding modules, barcode readers, filling modules, sampling modules, incubation modules, sensor modules (e.g., for determining cell density, cell viability, pH, oxygen concentration, nutrient concentration, fluorescence measurements, etc.), assay modules (e.g., for ELISA or other biological assays), data analysis and management modules, control modules, etc. Sensors, control systems, and the like may also be positioned to facilitate operation of the device. Certain embodiments of the invention may be used, for example, to promote or optimize chemical synthesis or cell or biological growth, for instance, for the production of compounds such as drugs or other therapeutics.

### RELATED APPLICATIONS

This application is a continuation-in-part of co-pending U.S. patent application Ser. No. 10/457,017, filed Jun. 5, 2003, entitled “System and Method for Process Automation,” by Seth T. Rogers, et al.

### FIELD OF THE INVENTION

This invention generally relates to systems and methods for manipulating substrates such as cell culture and other biological, biochemical, or chemical substrates.

### BACKGROUND

A wide variety of reaction systems are known for the production of products of chemical and/or biochemical reactions. Chemical plants involving catalysis, biochemical fermenters, pharmaceutical production plants, and a host of other systems are well-known. However, scale-up of chemical processes remains a difficult issue.

Biochemical processing can involve the use of a live microorganism (e.g., cells) to produce a substance of interest. Typically, cell cultures are performed in media suitable for cell growth and containing necessary nutrients. The cells are generally cultured in a location, such as an incubator, where environmental conditions can be controlled. Incubators traditionally range in size from small incubators (e.g., about 1 cubic foot) for a few cultures up to an entire room or rooms where the desired environmental conditions can be carefully maintained. Recently, as described in International Patent Application Serial No. PCT/US01/07679, published on Sep. 20, 2001 as WO 01/68257, entitled “Microreactors,” incorporated herein by reference, cells have also been cultured on a very small scale (i.e., on the order of a few milliliters or less), so that many cultures can be performed in parallel.

However, running large numbers of cell culture and other biological cultures using current techniques can be very labor- and material-intensive, for example during testing, development, or production. Additionally, the screening of various factors that might influence production can be costly and time-consuming, due to the large number of cultures that must be prepared, grown, and monitored, while varying numerous experimental parameters such as the specific cell lines, growth conditions, media, or the addition and timing of various chemical or biological agents.

### SUMMARY OF THE INVENTION

This invention generally relates to systems and methods for manipulating substrates such as cell culture chambers and other biological, biochemical, or chemical chips or substrates. The subject matter of this application involves, in some cases, interrelated products and/or uses, alternative solutions to a particular problem, and/or a plurality of different uses of a single system or article.

In one aspect, the invention is a system. In one set of embodiments, the system includes an apparatus constructed and arranged to secure a biological substrate. In some cases, the apparatus is independently able to rotate the biological substrate about an axis, and translationally move the biological substrate in at least one of a direction substantially perpendicular to the axis and a direction substantially parallel to the axis. In another set of embodiments, the system includes at least two modules, each able to perform a manipulation on a biological substrate, where the at least two modules are substantially radially arranged about an axis. The system, in yet another set of embodiments, includes a refrigeration module, and an automated apparatus constructed and arranged to secure a substrate and introduce the substrate into the refrigeration module. In still another set of embodiments of the invention, the system is defined, at least in part, by an apparatus constructed and arranged to secure a substrate and introduce the substrate into a sterilization module. In one set of embodiments, the system includes an apparatus constructed and arranged to secure a substrate exposed to an ambient environment, where the apparatus is able to, independently, rotate the substrate about an axis, and translationally move the substrate in at least one of a direction substantially perpendicular to the axis and a direction substantially parallel to the axis. In another set of embodiments, the system includes a module comprising a pH sensor, an oxygen sensor, a fluid transfer apparatus, and an imaging sensor.

In one set of embodiments, the system includes an apparatus constructed and arranged to secure a substrate that is exposed to an environment having at least about 10,000 particles/m³. In some instances, the apparatus is able to independently rotate the substrate about an axis, and translationally move the substrate in at least one of a direction substantially perpendicular to the axis and a direction substantially parallel to the axis.

The invention, in another aspect, includes a method. The method, in one embodiment, includes directing an apparatus to remove a biological substrate from a first module able to perform a manipulation on the biological substrate, rotating at least a portion of the substrate about an axis, and directing the apparatus to position the biological substrate in a second module able to perform a manipulation on the biological substrate.

The method, in another embodiment, includes acts of subjecting at least two predetermined reaction sites, each having a volume of less than about 1 ml, each to a different environmental condition, selecting an environmental condition having a desired effect on a species at a reaction site, and applying the selected environmental condition in a reactor, chip, or substrate containing cells.

In yet another embodiment, the method includes subjecting at least one biological substrate to a plurality of different environmental conditions, using an apparatus constructed and arranged to secure a substrate, where the apparatus is able to independently rotate the substrate about an axis.

The method includes, in still another embodiment, placing a plurality of cell types in a plurality of reactors or a plurality of chips, where the plurality of reactors or chips comprise a plurality of predetermined reaction sites having a volume of less than about 1 ml, subjecting the predetermined reaction sites to a range of environmental conditions, determining a response of the cell types to the environmental condition, and selecting at least one cell type from the plurality of cell types based on the response.

In another aspect, the invention is directed to a method of making an apparatus able to manipulate a substrate such as a biological, biochemical, and/or chemical substrate, e.g., as described in any of the embodiments herein. In yet another aspect, the invention is directed to a method of using an apparatus able to manipulate a substrate such as a biological, biochemical, and/or chemical substrate, e.g., as described in any of the embodiments herein. In still another aspect, the invention is directed to a method of promoting fabricating, selling, and/or using an apparatus able to manipulate a substrate such as a biological, biochemical and/or chemical substrate, e.g., as described in any of the embodiments herein.

Other advantages and novel features of the invention will become apparent from the following detailed description of various non-limiting embodiments of the invention when considered in conjunction with the accompanying drawings, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In cases where the present specification and a document incorporated by reference include conflicting disclosure, the present specification shall control. If two (or more) applications incorporated by reference include conflicting and/or inconsistent disclosure with respect to each other, then the later-filed application shall control.

### BRIEF DESCRIPTION OF THE DRAWINGS

Non-limiting embodiments of the present invention will be described by way of example with reference to the accompanying drawings in which:

FIG. 1 shows a top view of a system for manipulating a chemical, biological, and/or biochemical sample, according to one embodiment of the invention;

FIG. 2 shows a top view of a system according to another embodiment of the invention, including a plurality of handling devices;

FIG. 3 shows a side view of apparatus including a device constructed to manipulate one or more samples in relation to a plurality of modules, according to one embodiment of the invention;

FIGS. 4A-4D show various arrangements of system modules, according to various embodiments of the invention;

FIG. 5 shows a holding module according to an embodiment of the invention;

FIGS. 6A-6D shows a sensing module according to one embodiment of the invention; and

FIGS. 7A-7D shows a fluid transfer module according to another embodiment of the invention.

### DETAILED DESCRIPTION

Each of the following commonly-owned applications directed to related subject matter and/or disclosing methods and/or devices and/or materials useful or potentially useful for the practice of the present invention is incorporated herein by reference: U.S. Provisional Patent Application Ser. No. 60/188,275, filed Mar. 10, 2000, entitled “Microreactor,” by Jury, et al.; U.S. patent application Ser. No. 09/707,852, filed Nov. 7, 2000, entitled “Microreactor,” by Jury, et al.; International Patent Application No. PCT/US01/07679, filed Mar. 9, 2001, entitled “Microreactor,” by Jury, et al., published as WO 01/68257 on Sep. 20, 2001; U.S. Provisional Patent Application Ser. No. 60/282,741, filed Apr. 10, 2001, entitled “Microfermentor Device and Cell Based Screening Method,” by Zarur, et al.; U.S. patent application Ser. No. 10/119,917, filed Apr. 10, 2002, entitled “Microfermentor Device and Cell Based Screening Method,” by Zarur, et al., published as 2003/0077817 on Apr. 24, 2003; International Patent Application No. PCT/US02/11422, filed Apr. 10, 2002, entitled “Microfermentor Device and Cell Based Screening Method,” by Zarur, et al., published as WO 02/083852 on Oct. 24, 2002; U.S. Provisional Patent Application Ser. No. 60/386,323, filed Jun. 5, 2002, entitled “Materials and Reactors having Humidity and Gas Control,” by Rodgers, et al.; U.S. Provisional Patent Application Ser. No. 60/386,322, filed Jun. 5, 2002, entitled “Reactor Having Light-Interacting Component,” by Miller, et al.; U.S. patent application Ser. No. 10/223,562, filed Aug. 19, 2002, entitled “Fluidic Device and Cell-Based Screening Method,” by Schreyer, et al.; U.S. Provisional Patent Application Ser. No. 60/409,273, filed Sep. 9, 2002, entitled “Protein Production and Screening Methods,” by Zarur, et al.; U.S. patent application Ser. No. 10/457,048, filed Jun. 5, 2003, entitled “Reactor Systems Responsive to Internal Conditions,” by Miller, et al.; U.S. patent application Ser. No. 10/456,934, filed Jun. 5, 2003, entitled “Systems and Methods for Control of Reactor Environments,” by Miller, et al.; U.S. patent application Ser. No. 10/456,133, filed Jun. 5, 2003, entitled “Microreactor Systems and Methods,” by Rodgers, et al.; U.S. patent application Ser. No. 10/457,049, filed Jun. 5, 2003, entitled “Materials and Reactor Systems having Humidity and Gas Control,” by Rodgers, et al,. published as 2004/0058437 on Mar. 25, 2004; International Patent Application No. PCT/US03/17816, filed Jun. 5, 2003, entitled “Materials and Reactor Systems having Humidity and Gas Control,” by Rodgers, et al., published as WO 03/103813 on Dec. 18, 2003; U.S. patent application Ser. No. 10/457,015, filed Jun. 5, 2003, entitled “Reactor Systems Having a Light-Interacting Component,” by Miller, et al., published as 2004/0058407 on Mar. 25, 2004; International Patent Application No. PCT/US03/18240, filed Jun. 5, 2003, entitled “Reactor Systems Having a Light-Interacting Component,” by Miller, et al., published as WO 03/104384 on Dec. 18, 2003; U.S. patent application Ser. No. 10/457,017, filed Jun. 5, 2003, entitled “System and Method for Process Automation,” by Rodgers, et al.; U.S. patent application Ser. No. 10/456,929, filed Jun. 5, 2003, entitled “Apparatus and Method for Manipulating Substrates,” by Zarur, et al.; U.S. patent application Ser. No. 10/633,448, filed Aug. 1, 2003, entitled “Microreactor,” by Jury, et al.; International Patent Application No. PCT/US03/25956, filed Aug. 19, 2003, entitled “Determination and/or Control of Reactor Environmental Conditions,” by Miller, et al., published as WO 2004/016727 on Feb. 26, 2004; U.S. patent application Ser. No. 10/664,046, filed Sep. 16, 2003, entitled “Determination and/or Control of Reactor Environmental Conditions,” by Miller, et al.; International Patent Application No. PCT/US03/25907, filed Aug. 19, 2003, entitled “Systems and Methods for Control of pH and Other Reactor Environmental Conditions,” by Miller, et al., published as WO 2004/016729 on Feb. 26, 2004; U.S. Patent Application Ser. No. 60/498,981, filed Aug. 29, 2003, entitled “Rotatable Reactor Systems and Methods,” by Zarur, et al.; U.S. Patent Application Ser. No. 60/499,124, filed Aug. 29, 20003, entitled “Reactor with Memory Component,” by Zarur, et al.; U.S. patent application Ser. No. 10/664,068, filed Sep. 16, 2003, entitled “Systems and Methods for Control of pH and Other Reactor Environmental Conditions,” by Miller, et al.; International Patent Application No. PCT/US03/25943, filed Aug. 19, 2003, entitled “Microreactor Architecture and Methods,” by Rodgers, et al.; a U.S. Patent Application filed on Sep. 16, 2003, entitled “Microreactor Architecture and Methods,” by Rodgers, et al.; a U.S. Patent Application filed on Jun. 7, 2004, entitled “Control of Reactor Environmental Conditions,” by Rodgers, et al.; an International Patent Application filed on Jun. 7, 2004, entitled “System and Method for Process Automation,” by Rodgers, et al.; a U.S. Patent Application filed on Jun. 7, 2004, entitled “Apparatus and Method for Manipulating Substrates,” by Zarur, et al.; an International Patent Application filed on Jun. 7, 2004, entitled “Apparatus and Method for Manipulating Substrates,” by Zarur, et al.; a U.S. Patent Application filed on Jun. 7, 2004, entitled “Reactor with Memory Component,” by Zarur, et al.; an International Patent Application filed on Jun. 7, 2004, entitled “Reactor with Memory Component,” by Zarur, et al.; a U.S. Patent Application filed on Jun. 7, 2004, entitled “Gas Control in a Reactor,” by Rodgers, et al.; a U.S. Design Patent Application filed on Jun. 7, 2004, entitled “Reactor and Chip,” by Russo, et al.; a U.S. Patent Application filed on Jun. 7, 2004, entitled “Reactor Mixing” by Johnson, et al.; and a U.S. Patent Application filed on Jun. 7, 2004, entitled “Reactor Mixing Apparatus and Method,” by MacGregor.

This disclosure generally relates to systems and methods for manipulating chemical, biological, and/or biochemical samples, optionally supported on substrates and/or within chambers, for example biological samples contained on chips, within biological chambers, etc. In one aspect, the invention includes a system able to position a chamber or other substrate in one or more modules addressable by one or more handling or manipulating apparatuses, where the modules are positioned to be conveniently addressable by the handling apparatus, for example, positioned so as to surround the apparatus. The apparatus may be able to move the chamber or other substrate in any set of directions, such as radially, vertically, and/or rotationally, with respect to the apparatus itself, and may be manually operated and/or automatically controlled.

Examples of modules that can be part of such systems include, but are not limited to, sensing modules for determining the presence and/or a characteristic of a sample, actuating modules for physically manipulating (e.g., agitating) a sample and/or creating a particular environment for the sample (e.g., temperature), storage modules for aging samples and/or storing samples between other activities involving other modules, or introduction or completion modules for introducing samples into and removing samples from the system. Specific examples including modules for storing, stacking or holding modules, barcode readers, filling modules, sampling modules, memory and/or date-recording modules, incubation modules, sensor modules (e.g., for determining cell density, cell viability, pH, oxygen concentration, nutrient concentration, fluorescence measurements, etc.), assay modules (e.g., for ELISA or other biological assays), data analysis and management modules, control modules, etc. Sensors, control systems, and the like may also be positioned to facilitate operation of the device. Certain embodiments of the invention may be used, for example, to promote or optimize chemical synthesis or cell or biological growth, for instance, for the production of compounds such as drugs or other therapeutics.

In one embodiment, the invention includes a system comprising a cluster tool-type apparatus adapted to manipulate biological species including, but not limited to, cells. In other embodiments, the apparatus may be adapted to manipulate chemical samples, biochemical samples, or the like. A “cluster tool,” as used herein, is a device that can move objects between different locations, typically “modules,” where the objects are stored and/or subject to different testing and/or treatment conditions. Cluster tools may include a central, automated actuator that can rotate about a vertical axis, surrounded radially by modules into which and from which objects can be introduced and removed for various treatment steps in, e.g., circuit fabrication. In some cases, the central actuator may be an articulated arm, e.g., having one or more joints. As used herein, “automated” devices refer to devices that are able to operate without human direction. That is, an automated device can perform a function during a period of time after a human has finished taking any action to promote the function, e.g. by entering instructions into a computer. Typically, automated equipment can perform repetitive functions after this point in time.

The present invention, in certain embodiments, involves the adaptation of a cluster tool-type arrangement for subjecting biological, chemical, and/or biochemical samples to various environments in various modules, i.e., a cluster tool-type arrangement in combination with modules constructed and arranged to manipulate biological samples and/or introduce or remove biological samples from a sample chamber or other substrate. Those of ordinary skill in the art will understand, from the description that follows, that the invention can be practiced in a variety of ways, with a variety of arrangements that allow transport of a biological, chemical, and/or biochemical species, and/or substrates for supporting such a species, between and among various modules that can subject the species and/or substrate to various conditions, optionally using automated equipment.

As used herein, the term “determining” generally refers to the measurement and/or analysis of a species, for example, quantitatively or qualitatively, and/or the detection of the presence or absence of the species. The species may be, for example, a chamber or other substrate, a cell within a chamber, a compound within a chamber, etc. “Determining” may also refer to the measurement and/or analysis of an interaction between two or more species (for instance, two compounds, a compound and a cell, two cells, a cell and a chamber containing the cell, etc.), for example, quantitatively and/or qualitatively, and/or by detecting the presence or absence of an interaction.

As used herein, “secure” means to affix an object to an apparatus such that the object will not be dislodged from the apparatus due to motion of the apparatus. For example, the apparatus may invert, rotate, revolve, agitate, stir, and/or vibrate the object without dislodging it. The object, of course, may be intentionally removed from the apparatus by an operator (e.g., a mechanical or automated device, or a human user). As one example, a chamber or other substrate may be placed into a slot of an apparatus designed to secure the chamber or other substrate during use of the apparatus. For instance, a chamber (or other substrate) may be inserted into an apparatus in a slot designed to hold the chamber, thereby securing the chamber within the apparatus. Optionally, mechanical restraints, such as hooks, guides, clips, fasteners, bands, or springs may be used to secure the chamber or other substrate to the apparatus. As another example, a chamber (or other substrate) may be secured to an apparatus via a clamp. As yet another example, a chamber (or other substrate) may be secured in an apparatus in such a way that the chamber is able to move within the apparatus in some fashion without being dislodged from the apparatus due to motion of the apparatus.

As used herein, “sample” means a portion of a chemical, biological, and/or biochemical species, living or non-living, organic or inorganic, that is desirably manipulated in some fashion, for example, in the context of environmental control, motion (e.g., agitation), and/or the passage of time, etc. For example, a sample can be something desirably studied in terms how a particular environment or environments, motion, and/or time affects it; a sample can be a reactant, or starting material that is known to change chemically or biologically in response to a particular environment(s), motion, and/or time, which change is promoted via embodiments of the invention; a combination of these, or the like.

As used herein, a “substrate” is an article having a surface in and/or on and/or proximate to which a biological, biochemical, or chemical reaction can take place. A substrate may be planar or substantially planar, although in some cases, the substrate may be curved or otherwise non-planar, depending on the specific application. Non-limiting examples of materials useful for forming substrates can include glass, plastic, semiconductor materials, or the like. In some cases, the substrate may be modified to promote or inhibit certain reactions. For example, the substrate may be etched or coated with a chemical that enhances the hydrophobicity or hydrophilicity of the substrate, enhances the cytophobicity or cytophilicity of the substrate, promotes specific or non-specific binding of a reactant to or proximate the substrate, etc. The substrate may be at least partially enclosed in certain embodiments (e.g., as part of a chamber, or contained within a chamber), for example, as in a flask or an enclosed microfluidic system. In some cases, a reaction on a substrate may be altered in some fashion by the addition of a fluid, for example by causing or preventing a reaction in and/or on and/or proximate to the substrate, and/or promoting or inhibiting such reaction. A “chamber,” as used herein, is an article having or containing a substrate, and in some cases, may enclose or at least partially enclose the substrate. For example, the chamber may enclose a substrate therein, a substrate may define a wall of the chamber, etc.

A “biological substrate,” as used herein, is an article having a surface in and/or on and/or proximate to which a biological reaction can take place. A “biological chamber” is an article having or containing a substrate (e.g., as part of a chamber, or contained within a chamber) in which a biological system can be grown in vitro, for example, cells, tissue and tissue constructs, ex vivo systems, organisms, and the like. A biological chamber typically is enclosed or at least partially enclosed. The chamber may be formed out of any suitable material able to contain cells or other biological systems and/or may include a substrate that cells or other biological systems can adhere to, for example, a substrate comprising glass, polystyrene, and/or other materials known to those of ordinary skill in the art. A “cell culture chamber” is a biological chamber in which cells can be grown in vitro. The substrate typically is planar. Cell culture chambers are well-known in the art and include, but are not limited to, petri dishes (having any suitable diameter), flasks (e.g., T25 flasks, T75 flasks, T150 flasks, T175 flasks, etc.), microplates such as those defined in the 2002 SPS/ANSI proposed standard (e.g., a microplate having dimensions of roughly 127.76±0.50 mm by 85.48±0.50 mm), for example, 6-well microplates, 24-well microplates, 96-well microplates, etc.), and the like. The cell culture chamber may be formed out of any suitable material able to contain cells and allow cell culture to occur, for example, glass, polystyrene and/or other polymers, and/or materials known to those of ordinary skill in the art. In some cases, the cell culture chamber may be disposable.

One example of a biological chamber is a microplate. A “microplate” is also sometimes referred to as a “microtiter” plate, a “microwell” plate, or other similar terms known to the art. The microplate can have standardized or art-recognized dimensions, for example, as defined in the 2002 SPS/ANSI proposed standard (e.g., a microplate having dimensions of roughly 127.76±0.50 mm by 85.48±0.50 mm). The microplate may include any number of wells. For example, as is typically used commercially, the microplate may be a six-well microplate, a 24-well microplate, a 96-well microplate, a 384-well microplate, or a 1,536-well microplate. The wells may each be of any suitable shape, for example, cylindrical or rectangular. The microplate may also have other numbers of wells and/or other well geometries or configurations, for instance, in certain specialized applications.

In certain aspects of the invention, the cell culture or other biological chamber (or other substrate) may be substantially “watertight,” i.e., the chamber or substrate may be constructed and arranged such that a liquid inside the chamber or substrate, such as water, does not come out of the chamber or substrate regardless of the chamber's or substrate's orientation or position. For example, if the chamber is a flask, the flask may have a screw-on cap that can be attached to the flask to prevent liquids from coming out. As another example, the chamber may be a chip, for example, a sealed microplate, optionally with internal access to the microplate through self-sealing ports able to allow internal access, for example, when punctured with a needle. Non-limiting examples of self-sealing materials suitable for use with the invention include, for example, polymers such as polydimethylsiloxane (“PDMS”), or silicone materials such as Formulations RTV 108, RTV 615, or RTV 118 (General Electric, New York, N.Y.).

In embodiments in which a cell culture chamber is used, it may include a substrate suitable for growing a cell type that can be cultured in vitro, for example, a bacterium or other single-cell organism, a plant cell, or an animal cell. If the cell is a single-cell organism, then the cell may be, for example, a protozoan, a trypanosome, an amoeba, a yeast cell, algae, etc. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a mammalian cell such as a primate cell, a bovine cell, a horse cell, a porcine cell, a goat cell, a dog cell, a cat cell, or a cell from a rodent such as a rat or a mouse. If the cell is from a multicellular organism, the cell may be from any part of the organism. For instance, if the cell is from an animal, the cell may be a cardiac cell, a fibroblast, a keratinocyte, a heptaocyte, a chondracyte, a neural cell, a osteocyte, a muscle cell, a blood cell, an endothelial cell, an immune cell (e.g., a T-cell, a B-cell, a macrophage, a neutrophil, a basophil, a mast cell, an eosinophil), a stem cell, etc. In some embodiments, more than one cell type may be used simultaneously, for example, fibroblasts and hepatocytes. In certain embodiments, cell monolayers, tissue cultures or cellular constructs (e.g., cells located on a non-living scaffold), and the like may also be used. In some cases, the cell may be a genetically engineered cell. In certain embodiments, the cell may be a Chinese hamster ovarian (“CHO”) cell or a 3T3 cell. In some embodiments, more than one cell type may be used simultaneously, for example, fibroblasts and hepatocytes. In certain embodiments, cell monolayers, tissue cultures or cellular constructs (e.g., cells located on a non-living scaffold), and the like may also be used. The precise environmental conditions necessary for a specific cell type or types may be determined by those of ordinary skill in the art.

In some instances, the cells may produce chemical or biological compounds of therapeutic and/or diagnostic interest. For example, the cells may be able to produce products such as monoclonal antibodies, proteins such as recombinant proteins, amino acids, hormones, vitamins, drug or pharmaceuticals, other therapeutic molecules, artificial chemicals, polymers, tracers such as GFP (“green fluorescent protein”) or luciferase, etc. In one set of embodiments, the cells may be used for drug discovery and/or drug developmental purposes. For instance, the cells may be exposed to an agent suspected of interacting with the cells. Non-limiting examples of such agents include a carcinogenic or mutagenic compound, a synthetic compound, a hormone or hormone analog, a vitamin, a tracer, a drug or a pharmaceutical, a virus, a prion, a bacteria, etc. For example, in one embodiment, the invention may be used in automating cell culture to enable high-throughput processing of monoclonal antibodies and/or other compounds of interest.

Where a substrate is used, some portion or all of it may be treated in such a way as to promote attachment of cells or other biological cultures (i.e., a “biological substrate”). For example, a substrate may be ionized and/or coated with any of a wide variety of hydrophilic, cytophilic, and/or biophilic materials, for example, materials having exposed carboxylic acid, alcohol, and/or amino groups. In other embodiments, the surface of the substrate may be at least partially coated with a biological material that promotes adhesion, for example, fibronectin, laminin, vitronectin, albumin, collagen, and/or a peptide containing an RGD sequence. Other suitable hydrophilic, cytophilic, and/or biophilic materials will be known to those of ordinary skill in the art.

In certain embodiments, the substrate may be a substrate able to promote a chemical or a biochemical reaction, for example, in and/or on and/or proximate the substrate. The substrate may define one or more reactors and/or reaction sites. In some cases, the substrate may comprise a non-semiconductor material or a non-silicon material (i.e., a material that does not contain elemental silicon in semiconductor form). In certain embodiments, the substrate may also be contained or form a part of a chamber, for example, a cell culture or other biological chamber.

A substrate of the invention can include one or more reactors, which may each independently include one or more reaction sites. As used herein, a “reaction site” is defined as a site within a reactor that is constructed and arranged to produce a physical, chemical, biochemical, and/or biological reaction during use of the reactor. More than one reaction site may be present within a reactor or a substrate in some cases, for example, at least one reaction site, at least two reaction sites, at least three reaction sites, at least four reaction sites, at least 5 reaction sites, at least 7 reaction sites, at least 10 reaction sites, at least 15 reaction sites, at least 20 reaction sites, at least 30 reaction sites, at least 40 reaction sites, at least 50 reaction sites, at least 100 reaction sites, at least 500 reaction sites, or at least 1,000 reaction sites or more may be present within a reactor or a substrate. The reaction site may be defined as a region where a reaction is allowed to occur; for example, a reactor may be constructed and arranged to cause a reaction within a channel, one or more compartments, at the intersection of two or more channels, etc. The reaction may be, for example, a mixing or a separation process, a reaction between two or more chemicals, a light-activated or a light-inhibited reaction, a biological process, and the like. In some embodiments, the reaction may involve an interaction with light that does not lead to a chemical change, for example, a photon of light may be absorbed by a substance associated with the reaction site and converted into heat energy or re-emitted as fluorescence. In certain embodiments, the reaction site may also include one or more cells and/or tissues. Thus, in some cases, the reaction site may be defined as a region surrounding a location where cells are to be placed within the reactor, for example, a cytophilic region within the reactor.

As used herein, a “reactor” is the combination of components including a reaction site, any chambers (including reaction chambers and ancillary chambers), channels, ports, inlets and/or outlets (i.e., leading to or from a reaction site), sensors, actuators, processors, controllers, membranes, and the like, which, together, operate to contain, promote and/or monitor a biological, chemical, and/or biochemical reaction, interaction, operation, or experiment at a reaction site, and which can be part of a substrate, such as a chip. For example, a chip or substrate may include at least 5, at least 10, at least 20, at least 50, at least 100, at least 500, or at least 1,000 or more reactors. Examples of reactors include chemical or biological reactors and cell culturing devices, as well as the reactors described in International Patent Application No. PCT/US01/07679, filed Mar. 9, 2001, entitled “Microreactor,” by Jury, et al., published as WO 01/68257 on Sep. 20, 2001, incorporated herein by reference. Reactors can include one or more reaction sites or compartments. The reactor may be used for any chemical, biochemical, and/or biological purpose, for example, cell growth, pharmaceutical production, chemical synthesis, hazardous chemical production, drug screening, materials screening, drug development, chemical remediation of warfare reagents, or the like. For example, the reactor may be used to facilitate very small scale culture of cells or tissues. In one set of embodiments, a reactor of the invention comprises a matrix or substrate of a few millimeters to centimeters in size, containing channels with dimensions on the order of, e.g., tens or hundreds of micrometers. Reagents of interest may be allowed to flow through these channels, for example to a reaction site, or between different reaction sites, and the reagents may be mixed or reacted in some fashion. The products of such reactions can be recovered, separated, and treated within the reactor or substrate in certain cases.

A “chemical, biological, or biochemical reactor chip,” (also referred to, equivalently, simply as a “chip”) as used herein, is an integral article that includes one or more reactors. “Integral article” means a single piece of material, or assembly of components integrally connected with each other. As used herein, the term “integrally connected,” when referring to two or more objects, means objects that do not become separated from each other during the course of normal use, e.g., cannot be separated manually; separation requires at least the use of tools, and/or by causing damage to at least one of the components, for example, by breaking, peeling, etc. (separating components fastened together via adhesives, tools, etc.).

Many embodiments and arrangements of the disclosed devices are described with reference to a chip, or to a reactor, and those of ordinary skill in the art will recognize that the presently disclosed subject matter can apply to either or both. For example, a channel arrangement may be described in the context of one, but it will be recognized that the arrangement can apply in the context of the other (or, typically, both: a reactor which is part of a chip). It is to be understood that all descriptions herein that are given in the context of a reactor or chip apply to the other, unless inconsistent with the description of the arrangement in the context of the definitions of “chip” and “reactor” herein.

It should also be understood that the chips and reactors disclosed herein may have a wide variety of different configurations. For example, a chip may be formed from a single material, or the chip may contain more than one type of reactor, reservoir and/or agent. In some cases, a chip may contain more than one system able to alter one or more environmental factor(s) within one or more reaction sites within the chip. For example, the chip may contain a sealed reservoir and an upper layer that a non-pH-neutral gas is able to permeate across.

As used herein, a “channel” is a conduit associated with a reactor and/or a chip (within, leading to, or leading from a reaction site) that is able to transport one or more fluids specifically from one location to another, for example, from an inlet of the reactor or chip to a reaction site, e.g., as further described below. Materials (e.g., fluids, cells, particles, etc.) may flow through the channels, continuously, randomly, intermittently, etc. The channel may be a closed channel, or a channel that is open, for example, open to the external environment surrounding the reactor or chip containing the reactor. The channel can include characteristics that facilitate control over fluid transport, e.g., structural characteristics (e.g., an elongated indentation), physical/chemical characteristics (e.g., hydrophobicity vs. hydrophilicity) and/or other characteristics that can exert a force (e.g., a containing force) on a fluid when within the channel. The fluid within the channel may partially or completely fill the channel. In some cases the fluid may be held or confined within the channel or a portion of the channel in some fashion, for example, using surface tension (i.e., such that the fluid is held within the channel within a meniscus, such as a concave or convex meniscus). The channel may have any suitable cross-sectional shape that allows for fluid transport, for example, a square channel, a circular channel, a rounded channel, a rectangular channel (e.g., having any aspect ratio), a triangular channel, an irregular channel, etc. The channel may be of any size within the reactor or chip. For example, the channel may have a largest dimension perpendicular to a direction of fluid flow within the channel of less than about 1000 micrometers in some cases, less than about 500 micrometers in other cases, less than about 400 micrometers in other cases, less than about 300 micrometers in other cases, less than about 200 micrometers in still other cases, less than about 100 micrometers in still other cases, or less than about 50 or 25 micrometers in still other cases. In some embodiments, the dimensions of the channel may be chosen such that fluid is able to freely flow through the channel, for example, if the fluid contains cells. The dimensions of the channel may also be chosen in certain cases, for example, to allow a certain volumetric or linear flowrate of fluid within the channel. In one embodiment, the depth of other largest dimension perpendicular to a direction of fluid flow may be similar to that of a reaction site to which the channel is in fluid communication with. Of course, the number of channels, the shape or geometry of the channels, and the placement of channels within the chip can be determined by those of ordinary skill in the art.

While one reaction site may be able to hold and/or react a small volume of fluid as described herein, the technology associated with the invention also allows for scalability and parallelization. With regard to throughput, an array of many reactors and/or reaction sites within a chip or other substrate, or within a plurality of chips or substrates, can be built in parallel to generate larger capacities. Additionally, an advantage may be obtained by maintaining production capacity at the small scale of reactions typically performed in the laboratory, with scale-up via parallelization. It is a feature of certain embodiments of the invention that many reaction sites may be arranged in parallel within a reactor of a chip and/or within a plurality of chips. Specifically, at least five reaction sites can be constructed to operate in parallel, or in other cases at least about 7, about 10, about 50, about 100, about 500, about 1,000, about 5,000, about 10,000, about 50,000, or even about 100,000 or more reaction sites can be constructed to operate in parallel. In some cases, the number of reaction sites may be selected so as to produce a certain quantity of a species or product, or so as to be able to process a certain amount of reactant. Of course, the exact locations and arrangement of the reaction site(s) within the reactor, chip, or other substrate will be a function of the specific application.

In one set of embodiments, the chamber or other substrate may be a microfluidic chamber or substrate (e.g., a chamber or substrate having at least one fluidic pathway therein having a smallest cross-sectional dimension of less than about 1 mm). The microfluidic chamber may be sealed in some cases and/or define spaces that are enclosed such that the chamber can be inverted without releasing any liquids contained therein. Non-limiting examples of microfluidic chambers and other substrates include those disclosed in the U.S. and international patent applications incorporated by reference above.

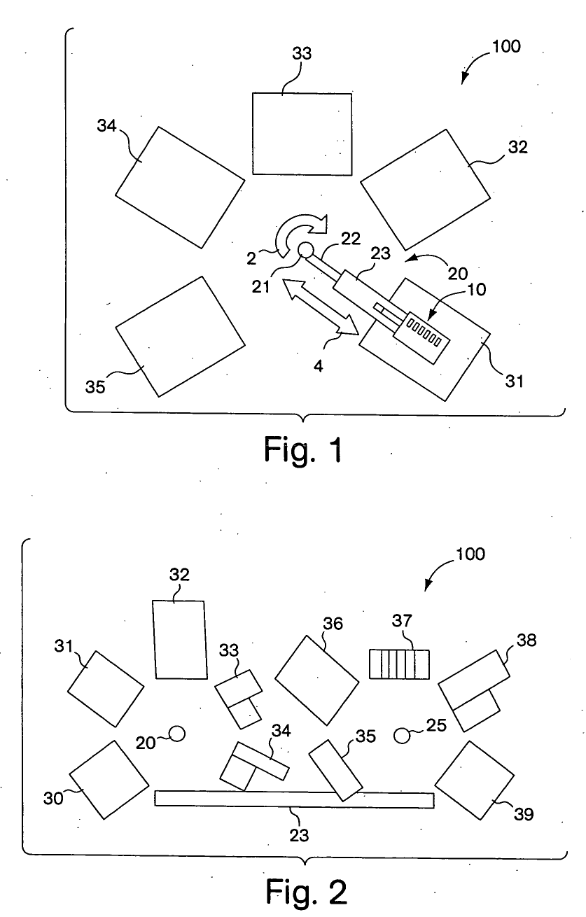

Referring now to the figures, in FIG. 1, system 100 includes handling apparatus 20, and a plurality of modules 31, 32, 33, 34, 35 positioned so as to be addressable by the handling apparatus (surrounding the handling apparatus in the embodiment illustrated). Handling apparatus 20 may be automated and/or under manual control. In FIG. 1, handling apparatus 20 includes a central pivoting mechanism 21 that pivots on a vertical axis, an arm 22 emanating from the central pivoting mechanism for addressing the various modules, and a sample securing mechanism 23 constructed and arranged to secure a sample and to introduce and/or remove the sample from at least one, and preferably all of the modules addressable by the apparatus. Securing mechanism 23 can be a clamp, a detent mechanism, a mechanism including protrusions insertable into corresponding indentations in a substrate or chamber containing the sample, or the like. As shown, a chamber 10 is secured by securing mechanism 23, and the system is able to move chamber 10 (or other substrate) about system 100. Pivoting mechanism 21 is able to rotate chamber 10 about an axis perpendicular to the plane of the paper (indicated by arrow 2, with the axis aligned with the center of mechanism 21), while arm 22 is able to move chamber 10 in a direction substantially perpendicular to the axis (i.e., in a radial direction towards or away from the axis, as indicated by arrow 4) and/or substantially parallel to the axis (i.e., in a direction perpendicular to the plane of the paper, direction not shown).

Radially arrayed around handling apparatus 20 are a series of modules 31, 32, 33, 34, 35. The modules are arranged such that handling apparatus 20 is able to add or remove a chamber to or from any of the modules. In FIG. 1, modules 31, 32, 33, 34, 35 are radially arranged in a substantially hexagonal arrangement around handling apparatus 20. Of course, in other embodiments, other arrangements of the modules may be utilized, as further discussed below. As illustrated in FIG. 1, arm 22 has been extended such that chamber 10 is positioned within module 31. If chamber 10 is to be removed from module 31, arm 22 can secure chamber 10 for removal by the arm. Conversely, if chamber 10 is to be positioned within module 31, then arm 22, which secures chamber 10, can release chamber 10 into the module. Similarly, handling apparatus 20 may be rotated and otherwise manipulated to move a chamber 10 into any of the other modules 32, 33, 34, 35 arranged about handling apparatus 20. Thus, as an example, handling apparatus 20 may remove chamber 10 from module 31 by extending an arm 22 to secure 10, retracting the arm and chamber 10, rotating via pivoting mechanism 21 to a direction so as to position chamber 10 within module 33, extending arm 22 to position chamber 10 within module 33, then releasing chamber 10 within module 33.

It should be noted that, although FIG. 1 illustrates a rotational apparatus able to, independently, rotate the chamber about an axis, and translationally move the chamber in at least one of a direction substantially perpendicular to the axis and a direction substantially parallel to the axis, that in other embodiments, other apparatuses may be used to move a chamber from one module to another, e.g., as further discussed below.

The handling apparatus may secure and/or transport one or more of the chambers and/or substrates to and from one or more modules located proximate the handling apparatus. The handling apparatus may manipulate the chambers or other substrates, for example, in response to a user or in an automated sequence. For instance, in FIG. 1, handling apparatus 20 can position a chamber in a first module (which may be any module accessible to handling apparatus 20), allow the module to perform an operation on the chamber (e.g., as described below) then move the chamber from the first module to a second module. The chamber can initially start, depending on the application, in a filled or partially filled state, or in an empty state (e.g., if the chamber will later be filled, for example, during the course of operation, optionally in a module). In one embodiment, the handling apparatus may include one or more effector mechanisms able to secure or “grab” chambers from a module, and/or position a chamber or other substrate within a module. Those of ordinary skill in the art will be able to chose appropriate mechanisms able to secure and/or position chambers or other substrates.

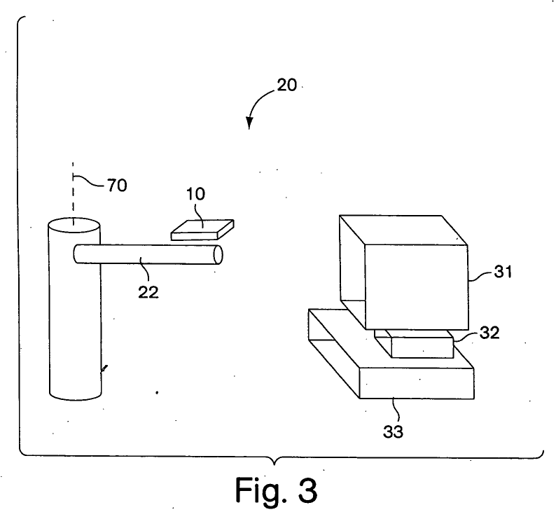

The handling apparatus may be able to move the chambers or substrates in two, three, or more axes or dimensions, for example, horizontally and vertically, or, in the case of a handling apparatus that is cylindrically coordinated, rotationally (e.g., about a substantially vertical axis), vertically, and/or radially. For instance, in FIG. 1, handling apparatus 20 is able to rotate and move chamber 10 radially, as indicated by arrows 2 and 4, respectively. As another example, the handling apparatus may include a multi-axis articulate automated robot having one or more arms sufficiently articulated so as to be able to retrieve and/or position chambers or other substrates within the modules. For example, the handling apparatus can include an automated “arm” having one or more articulated joints (for example, a shoulder, an elbow, and/or a wrist joint). As additional examples, the handling apparatus may include a cylindrical automated apparatus (e.g., as illustrated in FIG. 3), a linear translation stage, an elevator mechanism, a conveyor belt, etc.

In some embodiments, the inventive device may include more than one handling apparatus, for example, as illustrated in FIG. 2. In this figure, system 100 includes two handling apparatuses 20, 25, and a series of modules disposed around the two handling apparatuses. System 100 may be contained within a sterile environment, or within a non-sterile environment, such as in an ambient environment (e.g., air). In some cases, handling apparatus 20 can be configured to be able to directly transfer one or more chambers to handling apparatus 25 (or vice versa). In other cases, handling apparatus 20 can be configured to be able to indirectly transfer one or more chambers to handling apparatus 25 through a “hand-off” mechanism, such as, for example, a holding module or a conveyor belt, as is shown in FIG. 2.

In the example of FIG. 2, modules 30, 31, 32, 33 and 34 are arranged to be accessible handling apparatus 20, while modules 35, 36, 37, 38 and 39 are arranged to be accessible handling apparatus 25 (of course, in some embodiments, a module may be positioned so as to be simultaneously accessible to more than one handling apparatus, depending on the specific application, e.g., in a device in which one handling apparatus is configured to be able to “hand-off” a module to at least on other handling apparatus). Some of the modules (e.g., modules 33, 34, 37 and 38) can have more than one interior space, which space can be the same or different sizes. Modules 30, 31, 32, 33, 34, 35, 36, 37, 38 and 39 may be any suitable module for storing and/or manipulating a chamber, such as the modules described herein. For example, the modules may include a refrigeration module, a sterilization module, an incubation module, an assay module, a fluid transfer module, or module for providing an environmental condition or a range of environmental conditions, etc.

In FIG. 2, handling apparatuses 20 and 25 may be jointly or independently controlled and/or operated. In some cases, handling apparatuses 20 and 25 can be spaced sufficiently far apart that operation of handling apparatus 20 and handling apparatus 25 do not substantially interfere with each other. Thus, handling apparatus 20 and handling apparatus 25 can rotate and otherwise operate freely, without being able to contact each other. For instance, in FIG. 2, modules 33, 34, 35 and 36 are arrayed between handling apparatus 20 and handling apparatus 25, ensuring adequate separation between handling apparatus 20 and handling apparatus 25. In other embodiments, two or more handling apparatuses may be arranged such that they could come into contact, and the motions controlled such that undesired contact does not occur (of course, in some cases, certain contact may be desired, for example, to transfer a chamber from one handling apparatus to another).

In certain cases, transport of a chamber between the handling apparatuses may be desired. For instance, in FIG. 2, conveyor system 23 can be used to transport a chamber between handling apparatus 20 and handling apparatus 25. Thus, one end of conveyor system 23 is positioned such that handling apparatus 20 is able to position a chamber onto the conveyor system, while the other end of conveyor system 23 is positioned such that handling apparatus 25 is able to position or remove a chamber from the conveyor belt. Although in FIG. 2, the conveyor system is generally shown as linear, in other embodiments, the conveyor system does not necessarily have to be linear. For example, depending on the particular needs of an application, conveyor system may include bends, changes in slope or elevation, elevators, etc., as necessary to accommodate system 100 or a space (such as a room) containing system 100.

In some embodiments, the modules may also be vertically positioned relative to each other. For example, in FIG. 3, module 31 is vertically positioned on module 32, which is vertically positioned on module 33. Handling apparatus 20, containing arm 22 securing a chamber 10, is positioned to direct module 10 into any of modules 31, 32 and 33. For example, arm 22 can be extended to direct chamber 10 into a module and/or raised or lowered as desired. In addition, handling apparatus 20 may be rotated about axis 70 as desired.

The handling apparatus may move chambers or other substrates between modules in response to, for example, a program, instructions from a user, sensor measurements, etc. For instance, the handling apparatus(es) may be programmed to move chambers or substrates between modules using a control interface where a user inputs one or more desired operating parameters of the device. Examples of operating parameters include, but are not limited to, the type of cell or chemical reaction, a desired length of time, internal setpoints of the various modules, frequency of sensing, appropriate responses for various sensing measurements, frequency and type of fluids to be added, and the like. For instance, the handling apparatus may manipulate a chamber in response to a sensor measurement. Examples of sensors include proximity sensors, optical or visual sensors, temperature sensors, pressure sensors, or the like; other examples of sensors are further described below.

As one particular example, the handling apparatus may remove a chamber or other substrate, such as a cell culture chamber, from an incubator module (e.g., as further described below), place the chamber in a sensor or a sampling module, move the chamber from the module to a filling module, then move the chamber from the filling module back to the incubator module or to a second incubator module having different environmental conditions therein. This sequence of events may change, for instance, depending on the results of the measurements of the sensor or sampling module. For example, if the sensor or sampling module indicates that an environmental factor within the chamber is within established limits, then the handling apparatus may move the chamber directly to the incubator module instead of to a filling module. The particular methods used will depend on the specific application, and can be determined by those of ordinary skill in the art depending on the application.

The above-described modules may be arranged in such a way as to be accessible by the handling apparatus, i.e., such that the handling apparatus is able to add or remove at least one chamber (or other substrate) from the modules. As used herein, a “module” is an apparatus able to contain and/or perform a manipulation on a chamber or substrate. For example, the module may hold the chamber or substrate (e.g., for a finite period of time or under certain conditions or environments), heat and/or cool the chamber or substrate, determine the identity of a chamber or substrate (or a component or substance therein), perform a measurement on the chamber or substrate, add or remove a substance from the chamber or substrate, perform an assay on the chamber or substrate, control the pH of the chamber or substrate, allow a reaction and/or an interaction to occur within the chamber or substrate, etc. As additional non-limiting examples, in devices where cell culture chambers are used, the module may measure the concentration of one or more species within the cell culture chamber (such as oxygen, carbon dioxide, nitrogen, media, serum, ions, cells, etc.), determine an analyte, for instance as in a protein titer, an antibody titer, a cell titer, a hormone titer, a small molecule (i.e., a molecule having a molecular weight of less than about 1000 Da)titer, a product titer, a peptide titer, a ligand titer, etc. As another example, one or more characteristics of a cell and/or plurality of cells within the cell culture chamber may be determined, for example, cell concentration, cell density, cell viability, cell yield (e.g., of a product), cell productivity, cell type, cell morphology, cell adhesion, etc. Any of the above modules within the system can be replaced or substituted as desired, for example, to suit the needs of a particular application. Thus, in some cases, the modules are designed to be interchangeable. The modules may be replaced between operation cycles of the inventive system, and/or even while the system is being operated.

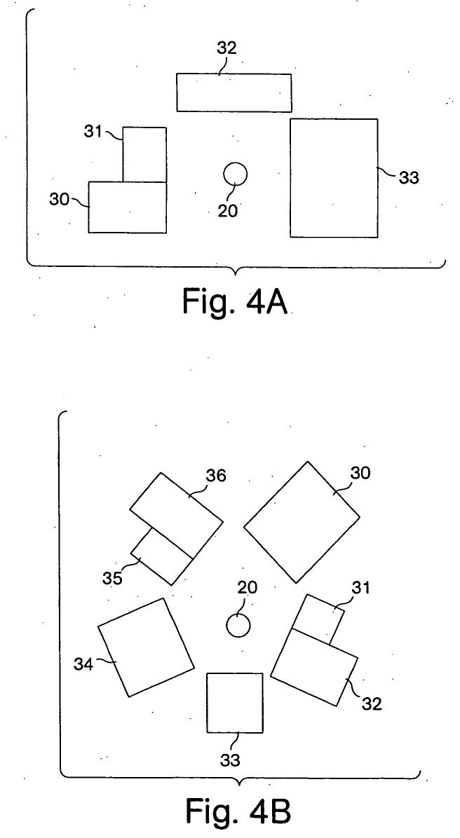

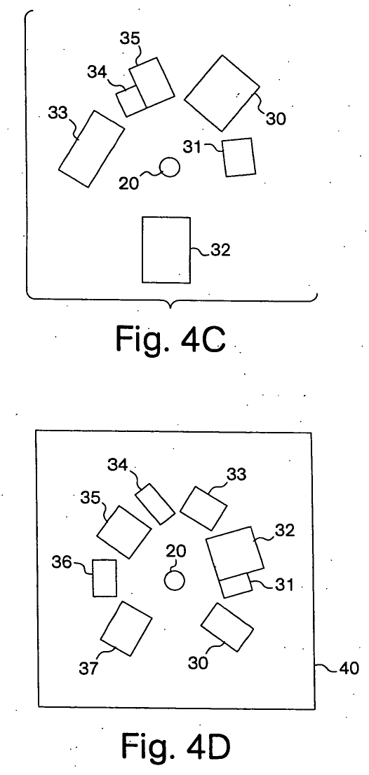

The modules may be arranged in any orientation such that the handling apparatus is able to access the modules. In some cases, the modules may be arranged substantially radially around a vertical axis, e.g., where a centrally-placed handling apparatus is used. For example, if a handling apparatus is centrally or substantially centrally positioned (e.g., as shown in FIGS. 1 and 4), then the modules may be arranged in any pattern such that the handling apparatus is able to access the modules. For example, the modules may be arranged in a circular or substantially circular pattern, or a polygonal or substantially polygonal pattern around the central handling apparatus. For instance, in FIG. 1, modules 31, 32, 33, 34, 35 are arranged in a substantially hexagonal pattern around handling apparatus 20, as discussed above. As used herein, “substantially polygonal” also includes embodiments where one or more sides of the polygon do not contain modules (e.g., as shown in FIG. 1).

Additional examples of substantially polygonal arrangements of modules about a handling apparatus are shown in FIGS. 2 and 4. In FIG. 2, modules 30 are arranged in a substantially hexagonal pattern around handling apparatus 20, and modules 35 are arranged in a substantially hexagonal pattern around handling apparatus 25. In FIG. 4A, modules 30, 31, 32, 33 are arranged around a handling apparatus 20. In this arrangement, the modules are arranged in a substantially square arrangement, with module 32 forming a second side of the substantially square arrangement, module 33 forming a third side of the substantially square arrangement, and modules 30, 31 forming one side of the substantially square arrangement (thus, more than one module may be present on a side). In FIG. 4B, a series of modules are arranged in a substantially pentagonal arrangement about handling apparatus 20; module 30 forms one side, modules 31 and 32 form a second side, module 33 forms a third side, module 34 forms a fourth side, and modules 35 and 36 form a fifth side of the substantially pentagonal arrangement. In FIG. 4C, a substantially heptagonal arrangement of modules around handling apparatus 20 is illustrated. In this figure, two of the seven sides of the heptagon do not have modules, while the remaining five sides of the heptagon contain modules 30, 31, 32, 33, 34 and 35. In FIG. 4D, an irregular arrangement of modules around handling apparatus 20 is shown. This arrangement illustrates a series of modules 30, 31, 32, 33, 34, 35, 36, 37 arranged in an irregular pattern (in this case, roughly circular) around handling apparatus 20. The handling apparatus and all of the modules in the example of FIG. 4D are additionally contained within housing 40. Housing 40 may be, for example, a housing able to keep dust and/or other particles from entering the apparatus, a housing able to maintain a controlled environment therein such as an incubator or a refrigerator, or a housing that can promote a clean and/or a biologically sterile environment therein.

Examples of modules that can be used with the invention include, but are not limited to: “stack” or “holding” module that can store or contain chambers (or other substrates), optionally in a sterile environment; a sterilization module able to sterilize the chambers (for example, through raising the temperature or the application of ionizing radiation); an identification module that can detect or determine specific chambers (for example, using identifying characteristics such as colors or bar codes, radio-frequency tags, or memory or other semiconductor chips); a data transfer module able to read or write data to or from a chamber; a fluid transfer module able to add and/or remove a substance to a chamber, for example cells, media, reagents, chemicals, pH buffers, initiators, etc.; a sensor module able to determine and/or record an environmental factor within the chamber, for example, pH, temperature, atmospheric conditions (e.g., gas concentrations), humidity, dissolved oxygen or carbon dioxide concentration, the concentration of other chemicals within the chamber (e.g., within the media), cell density, cell viability, cell morphology or other cell characteristics; an imaging module able to acquire an image of a chamber (e.g., optically, fluorescently, etc.); a refrigeration module; an incubation module able to maintain the temperature and/or other atmospheric conditions (such as the relative humidity) at a predetermined level; a sampling module able to remove a substance from a chamber (e.g., media, cells, products, etc.); an assay module able to perform chemical or biological assays on a chamber; or the like. Combinations of these and/or other modules are also envisioned, for example, a module that can fill and incubate a chamber or other substrate. Examples of these and other module functions are further described below.

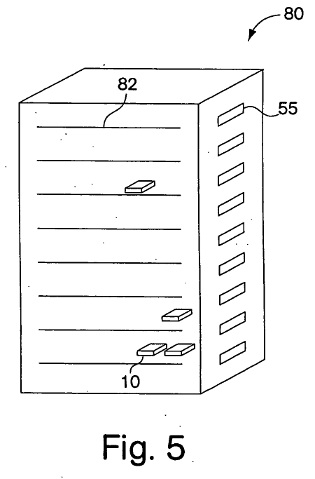

In one set of embodiments, at least one of the modules is able to hold or contain a chamber (or other substrate) for a certain length of time (e.g., a “holding” module, or a “stacking” module), for example, while the handling apparatus is manipulating other chambers, or where a certain amount of time is necessary before the chamber can be moved to the next step or the next module (e.g., when a reaction such as a chemical or an enzymatic reaction needs a certain amount of time to occur). The module can hold and/or secure one or more chambers, for example, as is shown by module 80 in FIG. 5. For example, the module can include one or more shelves or clamps able to hold and/or secure chambers, for example, shelves or clamps able to hold or secure microplates, flasks, roller bottles, etc. As one particular example, in FIG. 5, a rectangular module 80 contains a series of parallel horizontal shelves 82, at least some of which are able to contain one or more chambers 10. Module 80 is fully enclosed in this example, and access to the interior of module 80 is permitted only through a series of access ports 55, which are arranged such that each shelf 82 may be accessed directly through an access port 55. Access port 55 may be positioned anywhere within module 80 that allows suitable access of chambers or other substrates to module 80, for example, in a side of module 80, or on one or more major surfaces of module 80. For example, a chamber can be inserted through access port 50 into module 80 (e.g., onto a shelf 82, or other mechanical holding device). The chambers (or other substrates) can be inserted into and/or removed from module 80 via port 55 by essentially any technique including manual operation by hand, operation by an actuator, or operated by an automated actuator. Access port 55, in some embodiments, can be an opening in module 80, optionally including a flap, door, or other member that allows access port 55 to be closed when not being used to introduce or remove a chamber from module 80. In some cases, the module may be arranged such that a first handling apparatus is able to add a chamber to the module, while a second handling apparatus is able to remove the chamber from the module (of course, in other cases, each handling apparatus may be able to independently add and remove the chamber from the module). Such modules may also be referred to in some cases as “hand-off” modules.

As another example of a module function, a module may be able to identify one or more chambers (or other substrates) contained therein. In one embodiment, the module has an identification system, able to read an identification tag associated with a substrate or chamber such as a bar code, a serial number, a color tag, a radio tag, a magnetic tag, a radio-frequency tags, or memory or other semiconductor chips, or another identifying characteristic. For example, an identification tag, such as a bar code, may be etched or drawn on a chamber, or a sticker or other label containing an identifying tag may be affixed to the chamber. Suitable identification systems such as bar code readers or radio tags are generally available commercially. Other suitable identification systems include those disclosed in the U.S. and international patent applications incorporated by reference above. Thus, in some cases, the module may be able to determine if a chamber or a particular chamber is present in the module using the identification system, and react in an appropriate manner (for example, by recording the presence of the chamber, heating the chamber, performing an assay on it, causing a reaction to occur, etc.). In some cases, the module may use the identification system to track the movement or position of the chamber within the device, and/or to assist in data collection with regard to the chamber. The module may also transmit sensor or other data identified using the identification system to a processor for further analysis in some cases.

As yet another example of a module function, a module may be a data transfer module able to read and/or write data to the chamber (other substrate). For example, the data read and/or written to the chamber or substrate may include identification data, operating or storage condition data, results of assays or other manipulations to the chamber or substrate, etc. In one embodiment, the data may be written and/or read to a semiconductor chip or a magnetic medium integrally connected with the chamber or other substrate, for example, a memory chip, ROM chip, a magnetic tape, etc. Those of ordinary skill in the art know of suitable techniques for reading or writing data, e.g., to a semiconductor chip, a magnetic medium, etc. Additional examples of data transfer systems include those disclosed in the U.S. and international patent applications incorporated by reference above.

In another example of a function of a module, in some cases, a module may be able to determine and/or control the internal environment within the module (e.g., a “sensing” module), and/or within a chamber or substrate (or a portion thereof, such as within a reaction site). Determination and/or control of the environmental factor(s) within the module and/or within the chamber may be achieved, for example, using one or more sensors, processors, and/or actuators positioned on and/or in and/or proximate the module. Those of ordinary skill in the art will be able to determine, using no more than routine experimentation appropriate factors of the internal environment to be determined and/or controlled, depending on the application.

The sensor positioned in or proximate the module may be any suitable sensor able to determine an environmental factor associated with the module that will affect a biological species in the module, e.g., able to determine an environmental factor within the module, and/or able to determine an environmental factor within a chamber or other substrate or species itself. The sensor, in some cases, can determine one or more characteristics or environmental factors within the chamber, one or more reaction sites within the chamber, the contents of one or more reaction sites within the chamber (e.g., fluids or cells), etc. Examples of suitable sensors include, but are not limited to, an electrical sensor, a magnetic sensor, a proximity sensor, an optical or visual sensor, a spectroscopic sensor, a pH sensor, a mechanical sensor, a temperature sensor, a pressure sensor, a chemical sensor, a humidity sensor, a weight sensor, a sensor able to determine the concentration of a species etc. Appropriate sensors can be readily chosen or fabricated by those of ordinary skill in the art. In one embodiment, the module includes a sensor able to determine an environmental factor within a chamber containing cells. Non-limiting examples of such sensors include sensors able to determine the cell density (in 2 and/or 3 dimensions), cell viability, pH, dissolved oxygen concentration, dissolved carbon dioxide concentration, nutrient concentration, temperature, pressure, relative humidity, or the like. The sensor(s) may be embedded and integrally connected within the module, or separate from the module but able to determine environmental or other factors as discussed above (e.g., an optical sensor in optical communication with a module, or chamber or other substrate). Accordingly, the sensor may include an optical and/or a visual detector. Examples of suitable optical sensors include, but are not limited to, light-scattering, light absorption, optical density, polarization, light emission, fluorescence, and spectroscopic detectors. Examples of sensors able to perform suitable optical measurements include, but are not limited to, charge-coupled device (CCD) chips or cameras, photomultiplier tubes (PMT tubes), photodiodes such as avalanche photodiodes or photodiode arrays, photodetectors, photovoltaic cells, etc. In some cases, the sensor may be able to detect light produced by the chamber or by a component within the chamber. The chamber (or other substrate) may, for example, include a reaction that generates photons. For instance, the chamber may include a chemical reaction that produces photons, such as a reaction involving GFP (“green fluorescence protein”) and/or luciferase, and/or the chamber may include compounds able to produce light through fluorescence or phosphorescence. For example, incident electrons, electrical current, friction, heat, chemical, and/or biological reactions may be applied to a compound within the chamber to generate light.

In one embodiment, the optical sensor is able to detect a material having a direct or indirect calorimetric or fluorescent response, for example, upon exposure of the material to a certain compound or a certain cell or type of cells. In some cases, the material may be incorporated into the chamber or substrate. For example, the material may be incorporated within the chamber such that it is in fluidic contact with a reaction site within the chamber, e.g., immobilized at the end of a fiber optic or waveguide (e.g., as disclosed in U.S. patent application Ser. No. 10/457,049, filed Jun. 5, 2003, entitled “Materials and Reactor Systems having Humidity and Gas Control,” by Rodgers, et al,. published as 2004/0058437 on Mar. 25, 2004, incorporated by reference herein). In other cases, the material is separate from the chamber. For example, the material may be added to the chamber in a previous operation (e.g., a tracer compound), or the material may be present within the chamber (e.g., dissolved in a liquid present within the chamber). In certain instances where a cell culture is present within the chamber, the material may be dissolved in the media surrounding the cells, internalized by the cells, adsorbed on the surface of the cells, etc.

In some cases, the optical sensor may be able to acquire an image (an “imaging sensor”), for example of a chamber or substrate, and/or a component within the chamber, such as a reaction site. The microscopic image may be, for example, an optical image, a fluorescent image, a phase contrast image, etc., and will depend on the particular application. In one set of embodiments, the microscopic image can be magnified at least about 5-fold, at least about 10-fold, at least about 20-fold, at least about 50-fold, or at least 100-fold or more, depending on the specific application. In some cases, the microscopic image may be recorded using a camera (for example, using a CCD chip or camera), and/or transferred to a processor for further processing. The optical sensor may thus allow counting and/or visual inspection of the chamber and/or a component of the chamber, for example, cells within a reaction site. As such an example, an imaging sensor may be able to determine the morphology of the cells, determine the number of cells and/or cell density within the chamber, determine the concentration of compound present within and/or proximate the cells, determine the viability of the cells, determine the response of a cell to an agent such as a drug, etc. In some cases, the imaging system can store the results of such images, for example, as a digital image, and/or send image data to a processor for further analysis.

In certain cases, a module may also include a light source able to interact with the chamber (or other substrate) and/or optical sensor e.g., such that the light is altered in some fashion by the chamber. For example, the sensor may detect the degree of light absorption, optical density, polarization, etc., caused by the chamber and/or a component within the chamber, and/or the sensor may be a camera able to capture an image of the chamber as described above. The light source may be, for example, external or ambient light, a coherent or monochromatic beam of light such as created in an LED, or a laser such as a semiconductor laser or a quantum well laser. Suitable light sources within or proximate the module include, but are not limited to, a light-emitting diode (LEDs), arc lamps, continuous wave lasers, pulse lasers, and the like. Light from the light source may be directed substantially towards the chamber directly, or by means of optical components such as mirrors, lenses, prisms, optical fiber, waveguides, beamsplitters, filters, polarizers, lenses, prisms, diffraction gratings, and the like. In one set of embodiments, the light is directed substantially at one or more reaction sites within a chamber or other substrate.

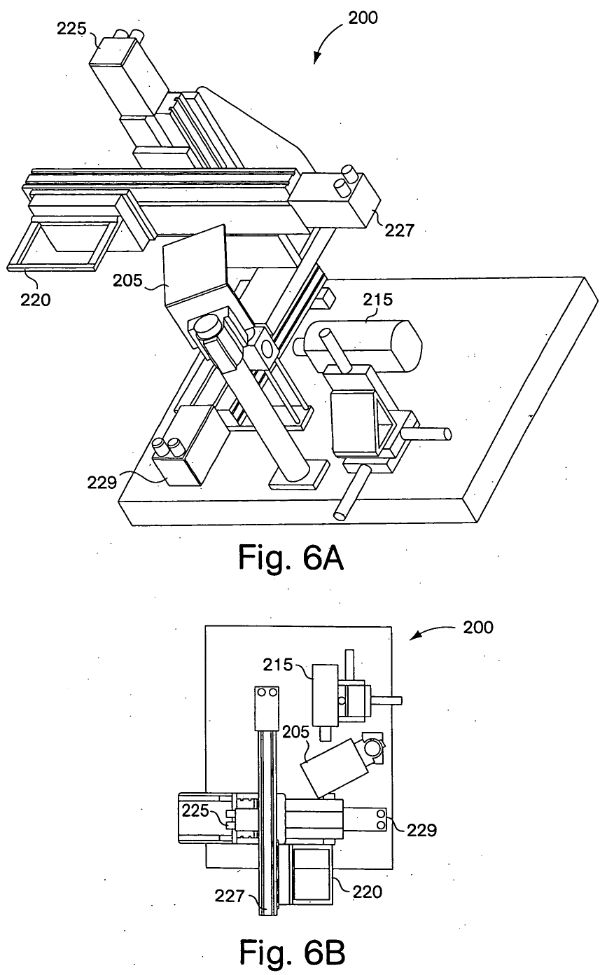

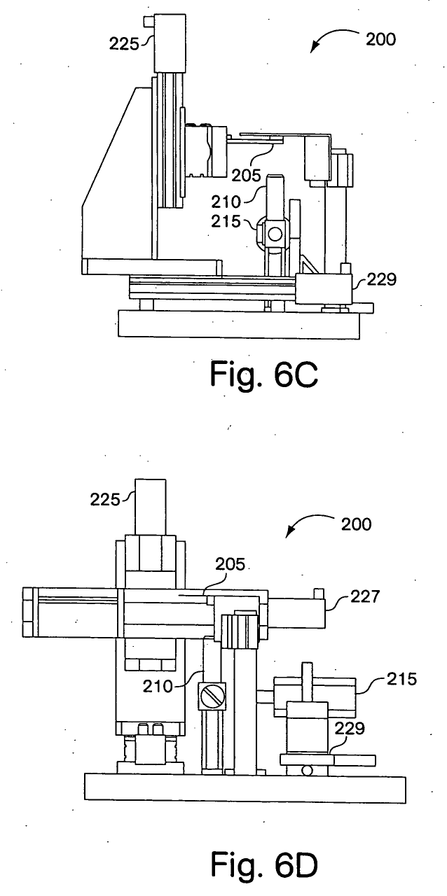

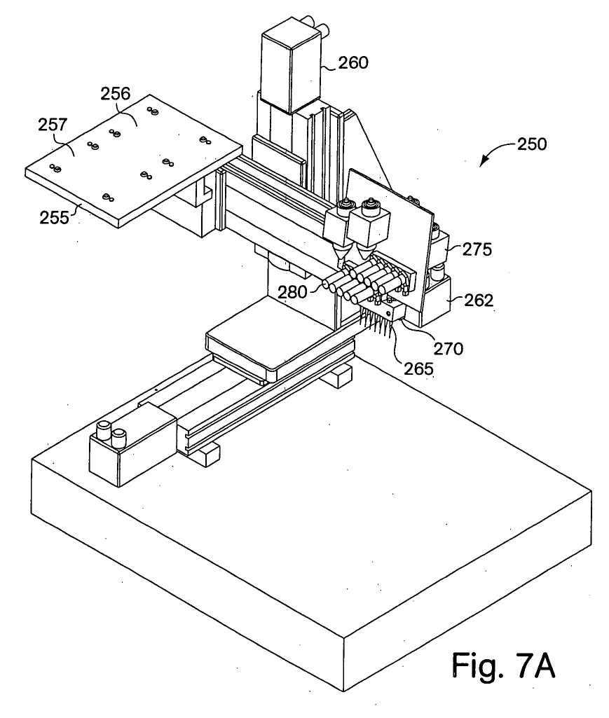

One non-limiting example of such a sensing module is shown in FIGS. 6A-6D (showing the module from various viewpoints). In these figures, sensing module 200 includes a light source 205 and an optical sensor. The optical sensor includes, in the example illustration, a microscope objective 210 (mounted on holder 229) coupled to a video camera 215. The video camera, in turn, may be coupled to a computer, an image processor, a video recorder, etc. Sensing module 200 also includes a positioning mechanism, including a sample holder 220 configured to be able to secure a chamber or other substrate, and translation mechanisms 225, 227 configured to be able to position the chamber or other substrate between light source 205 and microscope objective 210. In this embodiment, translation mechanisms 225, 227 are collectively configured to be able to independently manipulate the chamber in two directions. Thus, as an example, an apparatus containing a chamber or other substrate may position the chamber or substrate into sample holder 220. Translation mechanisms 225, 227 then operate to position the chamber or substrate in a position where a measurement or other determination can be made of the chamber or substrate by the optical sensor.

The sensor may be operatively connected to an actuator in some embodiments, and optionally to a processor. In some cases, one or more environmental factors within the module may be determined at regular intervals, and optionally, data related to the environmental factor(s) may be sent to a processor, or otherwise saved for further analysis. As used herein, a “processor” or a “microprocessor” is any component able to receive a signal from one or more sensors, store the signal, and/or convert the signal into one or more responses for one or more actuators, for example, by using a mathematical formula, and/or an electronic or computational circuit. In some cases, the processor can store data related to the signals for further analysis, for example, by downloading data related to the signals to a computer. The signal(s) from the sensor(s) may be any suitable signal, for example a pneumatic signal, an electronic signal, an optical signal, a mechanical signal, etc. The processor may be, for example, a mechanical apparatus, or an electronic device such as a semiconductor chip. The processor may be embedded and integrally connected with the module, or separate from the module, depending on the application. In one embodiment, the processor is programmed with a process control algorithm, which can, for example, take an incoming signal from a sensor and convert the signal into a suitable output for an actuator. Any suitable algorithm(s) may be used by the processor, for example, a PID control system, a feedback or feedforward system, a fuzzy logic control system, etc. The processor may be programmed or otherwise designed to control an environmental factor within the module, for example, by manipulation of an actuator.

As used herein, an “actuator” is a mechanism able to affect one or more environmental factors within and/or proximate the module. The actuator may be separate from, or integrally connected to the module. For example, in some embodiments, the actuator may include a valve or a pump configured to be able to control, alter, and/or prevent the flow of a species into and/or out of the module, for example, a chemical solution, a buffering solution, a gas such as CO₂ or O₂, a nutrient solution, a media component, or an acid or a base. The substance to be transported will depend on the specific application. As one example, the actuator may selectively open a valve that allows CO₂ or O₂ to enter the module. As another example, the actuator may include a pumping system that can create a fluid connection with a reaction site as necessary. As yet another example, the actuator may include a heating element and/or a cooling element, such as a resistive heater or a Peltier cooler. In yet another example where at least two fluid streams enter or leave a module, the actuator may include a valve or a pump that is able to control the ratio of flowrates between the two fluid streams. For instance, the actuator, in response to a signal, may act to increase an inlet flowrate and decrease an outlet flowrate to the module.

In one set of embodiments, the actuator includes an energy source for affecting an environment associated with a module, such as an electromagnetic energy source, a heat source, and/or an ultrasound source. In some embodiments, the electromagnetic radiation may have wavelengths or frequencies in the optical or visual range (e.g., having a wavelength of between about 400 nm and about 700 nm), infrared wavelengths (e.g., having a wavelength of between about 300 μm and 700 nm), ultraviolet wavelengths (e.g., having a wavelength of between about 400 nm and about 10 nm), or the like. In some cases, the light may cover a range of frequencies, for example, between about 350 nm and about 1000 nm, between about 300 μm and about 500 nm, between about 500 nm and about 1000 nm, between about 400 nm and about 700 nm, between about 600 nm and about 1000 nm, or between about 500 nm and about 450 nm. In other cases, the light may be monochromatic (i.e., having a single frequency or a narrow frequency distribution), for example, a frequency that is commonly produced by commercial lasers, or a frequency at which a fluorescent tracer is excited.

The sensor, actuator, and processor (if present) may thus form a control system that is configured to be able to control an environmental factor within the module and/or within a chamber or substrate, for example, to a predetermined setpoint, and/or in response to a certain condition. For example, the control system may, in some cases, allow internal control of one or more environmental factors within the module and/or within a chamber, such as the pressure, the concentration of one or more gases (e.g., the concentration oxygen, carbon dioxide, nitrogen, argon, helium, etc., either in the gaseous state or in solution), the relative humidity (for instance, greater than about 90%, greater than about 95%, or about 100% when a saturated environment is desired, or less than about 10%, less than about 5%, or about 0%, when a dehydrated environment is desired), etc. In another example, the control system is configured to be able to control the precise dispensing of fluids to and from a chamber or a component within a chamber, as further described below. In yet another example, the control system may be designed to be able to maintain a constant temperature within a module or chamber. For instance, the module may be refrigerated (e.g., as in a refrigerator or a freezer module) or heated (e.g., as in an “incubator” module). As one example, if the module contains cell culture chambers, the temperature of the module may be controlled at about 32° C., at about 35° C., or about 37° C., depending on the cell type. Thus, in one embodiment, the module is a cell culture incubator, for example, as disclosed in U.S. patent application Ser. No. 10/456,929, filed on Jun. 5, 2003, entitled “Apparatus and Method for Manipulating Substrates,” by Zarur, et al., incorporated by reference herein.

In still another example, the control system may measure and/or control the pH within a chamber or substrate, or a component within a chamber. For instance, the control system may include a sensor able to determine radiometric measurements of light absorption or emission from a fluorescein dye immobilized within a sol-gel matrix located within the chamber. The method may be useful in, for example, a high-throughput design. This method may not require calibration in some cases, and may require only a small-disposable sol-gel pellet to be in fluidic contact with the chamber and/or component within the chamber in certain instances. In some cases, the control system may be able to control the pH within the chamber and/or a component within the chamber to within about 1 pH unit, within about 0.5 pH units, within about 0.2 pH units, or within about 0.1 pH units. Control may be achieved, for instance, by comparing the pH of the chamber or component with a predetermined setpoint. An acid or a base may be applied to the chamber as necessary by the control system to control the pH, for example, by injecting the acid or base into or proximate the chamber or component.

In yet another example of a function of a module, a module may be able to add (e.g., a “filling” module”) or remove (e.g., a “sampling” module) a species to a chamber or substrate, or a component within the chamber, such as a reaction site. In some cases, the module may be able to both add and remove a species to a chamber or substrate. For example, the module may include a fluid transfer system able to add and/or remove a certain specified amount of chemicals, initiators, raw materials, liquids, cells, media, reagents, products, etc. to or from the chamber or a component thereof, such as a reaction site, e.g., in response to an actuator and/or a sensor. As another example, the fluid transfer system may remove a sample from the chamber, for example, for analysis or for further processing. If the chamber includes more than one inlet and/or outlet, the fluid transfer system may add or remove the species in any pattern from the inlets/outlets, for example, sequentially, randomly, simultaneously, etc. Techniques for adding or removing substances to and from a chamber are known to those of ordinary skill in the art. For instance, the module may include a fluid transfer system such as a needle, a pin, a pipette or micropipette, a syringe, etc., that enables a species to be introduced or withdrawn from the chamber. In some cases the transfer system may include a manifold, e.g., a system having a plurality of needles, pins, and/or pipettes. In one embodiment, the fluid transfer system is able to penetrate a septum or a self-sealing membrane in the chamber, or otherwise make fluidic contact with an interior of the chamber. The fluid transfer system may also be in fluidic communication with one or more sources of fluid, for example, a fluid reservoir, a source of water, a cell suspension, a gas cylinder, etc.

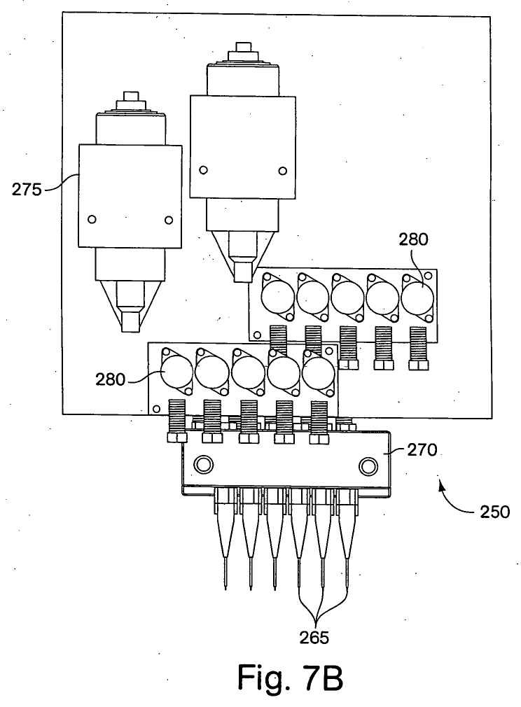

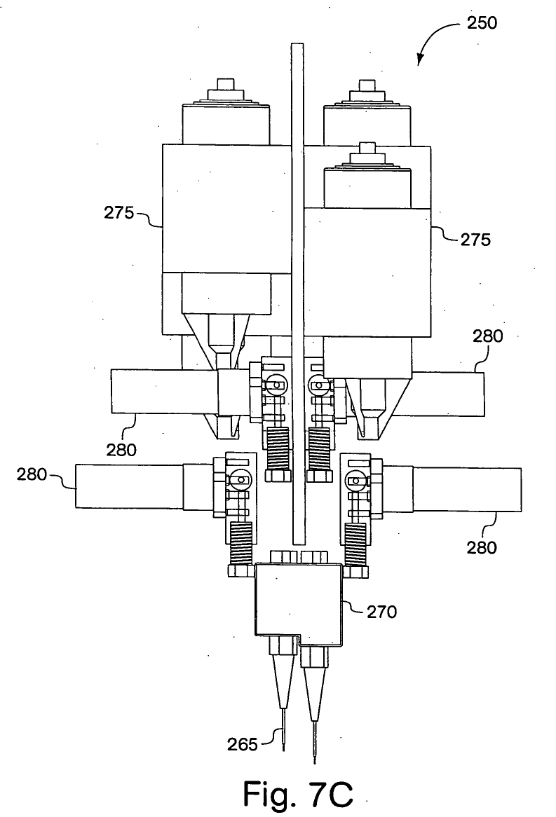

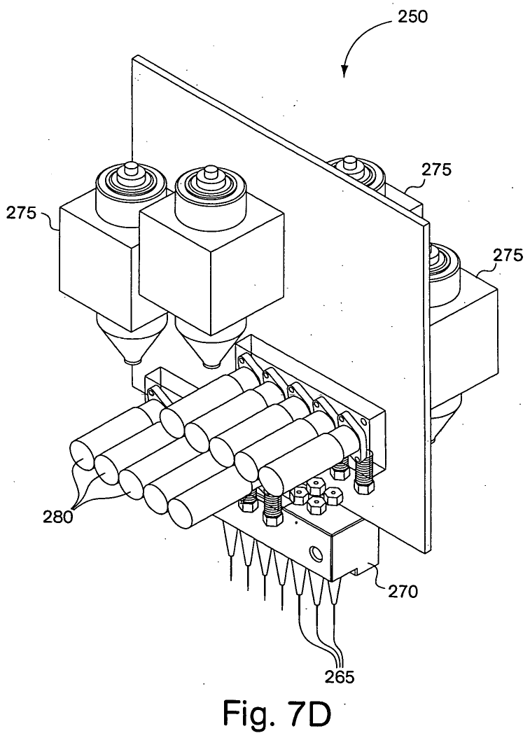

One non-limiting example of a fluid transfer module is illustrated in FIGS. 7A-7D. In these figures, fluid transfer module 250 includes sample holder 255, which is able to secure two chambers or other substrates in positions 256 and 257. Translation mechanisms 260, 262 can move, together or independently, in such a way as to move the chamber(s) to a location proximate needles 265 for fluid transfer. Needles 265 are connected to a needle manifold 270 (FIGS. 7B-7D show enlarged views of needle manifold 270 and the associated regions). Fluid to the needles is pumped by pumps 275 through valves 280 and needle manifold 270. Pumps 275 and valves 280 can be independently controlled to deliver fluid to, or extract fluid from, some or all of the needles as desired. For example, one needle may deliver fluid to, while a second needle may withdraw fluid from, a chamber or other substrate. Pumps 275 may be connected to another fluid chamber (not shown), e.g., a fluid reservoir, an assay system, a collection chamber, etc. In some embodiments, sample holder 255 can be operated such that a chamber or other substrate is positioned below needles 265, then raised upward such that needles 265 are able to enter the chamber in some fashion, e.g., through one or more ports. In other embodiments, fluid transfer module 250 may also include a mechanism able to raise or lower the needles into position, for example, to enter a chamber or substrate in some fashion, e.g., through one or more ports.

In some cases, a module may include a reservoir able to contain one or more fluids therein in fluidic communication with the fluid transfer system. The fluid transfer system can thus draw fluid from the reservoir and/or flow fluid into the reservoir. In some instances, one or more pumps, valves and/or other sources of pressure may enable fluid to be drawn from the reservoir to the fluid transfer system, or vice versa. The pumps and/or valves may be under the control of a processor and/or in communication with one or more sensors. In some cases, the pumps and/or valves may be chosen to minimize dead fluid volumes therein. In certain cases, the configuration of pumps, valves, tubing, connectors and other fluidic connections can be altered as desired, e.g., before or during operation. The reservoir may be maintained in a controlled environment in certain embodiments, e.g., a sterile environment, a temperature-controlled environment, a pressure-controlled environment, an environment where the relative humidity is controlled, etc. In certain cases, the reservoir and/or the fluid transfer system may also be able to be rinsed with a cleaning agent, for example, a detergent, bleach, ethanol, water, etc., to ensure sterility and/or cleanliness.

As one example of a fluid transfer system, the module may be able to add a chemical able to control pH to the chamber or other substrate (or a component within the chamber or substrate, such as a reaction site), for instance, in response to a pH determination e.g., from a sensor, as previously described. As another example, if the chamber is a cell culture chamber, the module may be able to initially fill the chamber (or reaction sites within the chambers), adjust the pH within the chamber, add cells and/or media to the chamber, add reagents to the chamber, add tracing agents to the chamber, etc. In one set of embodiments, the reservoir may include an initial cell culture or cultures (an “inoculum”), cell growth media, chemicals for maintaining pH, other reagents or initiators, etc., as required by a particular cell culture application. For example, an empty cell culture chamber may have added to it, within the module, an inoculum, cell growth media and/or other reagents, initiators, hormones, inducers, promoters, nutrients, stains, tracers, etc. The module may also be able to add or remove cells, media, or other chemical or biological fluids to and from the chamber.

In some instances, the fluid transfer system may be able to add or remove small amounts of material from the chamber or substrate, or component of the chamber, for example, products, cells, media, etc. For instance, the material added to the chamber may be unavailable in large amounts, or it may be desirable to dispense the materials to the chambers in a series of small doses, for example, in a predetermined schedule. In some cases, small volumes may be regularly removed from the chamber for analysis without significantly charging the volume of fluid within the reaction site, thereby allow multiple assays to be performed on the same chamber (or a component within the chamber, such as a reaction site) during the course of the experiment. Similarly, the addition of a concentrated reagent to the cell culture or other reaction site may be achieved without substantially altering the concentration of other materials within the chamber (or a component within the chamber, such as a reaction site).

In certain instances, the fluid transfer system is capable of adding or withdrawing very small volumes (i.e., less than about 1 ml or less than about 0.5 ml) of fluid with substantial accuracy and/or negligible dead volume. In some cases, the volume added or withdrawn may be less than about 300 microliters, less than about 100 microliters, less than about 30 microliters, less than about 10 microliters, less than about 3 microliters, less than about 1 microliter, less than about 300 nl, less than about 100 nl, less than about 30 nl, less than about 10 nl, less than about 3 nl, less than about 1 nl, less than about 300 pl, less than about 100 pl, less than about 30 p1, or less than about 10 pl in some cases. An example of such a fluid transfer system suitable for use with the invention is described in U.S. patent application Ser. No. 10/117,720, filed Apr. 4, 2002, entitled “System and Method for Dispensing Liquids,” by Kale, et al., incorporated herein by reference.

In another example of a function of a module, a module may include a sterilization system able to sterilize a chamber or other substrate, for instance to kill or otherwise deactivate biological cells (e.g., bacteria), viruses, etc. therein. The sterilization system may sterilize the chamber or substrate using chemicals, radiation (for example, with ultraviolet light and/or ionizing radiation), heat-treatment (e.g., raising the temperature above the boiling point of water), or the like. Appropriate sterilization techniques and protocols are known to those of ordinary skill in the art. For example, in one embodiment, the module is an autoclavable (e.g., a module configured to be able to raise the temperature to greater than about 100° C. or about 120° C., optionally at elevated pressures, such as at a pressure more than one atmosphere). Another exemplary sterilization system is a system configured to be able to expose the chamber or substrate to ozone.

In another example of a function of a module, a module may include a positioning system able to position a chamber or other substrate within the module. Those of ordinary skill in the art will know of suitable positioning systems for use within a module. For example, the positioning system may position a chamber or substrate within the module using a solenoid valve (which may be rotary) and/or a linear pneumatic actuator. The positioning system may be configured to position the chamber or substrate within the module, for example, at a predetermined location, in response to operator control, in response to sensor input, etc.

Combinations of the above functions and/or other functions may be included within a module. Thus, in one embodiment, the module is configured to be able to perform an assay on a chamber or substrate, or a component within the chamber or substrate, such as a reaction site, for example, using a combination of sensors, processors, control system, etc. For example, the module may be configured to perform a biological assay on the chamber (or on components within the chamber), such as an ELISA, an immunoassay, an affinity binding assay, a blotting assay, a spectrometric determination, a polarization determination, or the like. For instance, the module may be configured to be able to perform a biological, chemical, and/or biochemical assay automatically, in conjunction with monitoring or sensing of the chamber by a sensor. Those of ordinary skill in the art will readily envision other assays that can be adopted for use with the invention.

Design of the modules and the materials used for fabrication of the devices and components thereof will depend on the particular application and functionalities required. In some cases, the materials may be chosen based on factors such as the price or availability, resistance to degradation (e.g., at elevated or reduced temperatures, pressures, etc.), ease of cleaning, sterilization, use, replacement, etc. For example, the module (and/or substrate, or other component desirably sterilized) may include a sterilizable material. That is, the module or other component may be constructed from materials able to withstand treatments that can kill or otherwise deactivate biological cells (e.g., bacteria), viruses, etc. therein, before the module or substrate is used or re-used. For example, the component may be configured to be able to be sterilized with chemicals, radiated (for example, with ultraviolet light and/or ionizing radiation), heat-treated, or the like. Appropriate sterilization techniques and protocols are known to those of ordinary skill in the art. In one embodiment, the interior of a module is configured to be able to withstand autoclaving conditions (e.g., exposure to temperatures greater than about 100° C. or about 120° C., often at elevated pressures, such as pressures of more than one atmosphere), such that the module, after sterilization, does not substantially deform or otherwise become unusable. Another exemplary sterilization technique is exposure to ozone. In cases where a housing is used, e.g., as further described below, components contained within the housing (e.g., the handling apparatus and/or the module units) may also be sterilized.

As another example, a module may be formed from or include a metal able to withstand temperatures of at least about 100° C., for example, copper or stainless steel. Copper may be particularly useful in some embodiments, as copper may discourage the growth of some fungi. As yet another example, the material may include titanium or aluminum. In some cases, the module may be formed from other materials able to withstand temperatures of at least about 100° C. and/or other autoclaving conditions, for example, ceramics or certain polymers that may be heat-resistant. Other suitable materials can be readily selected by those of ordinary skill in the art.

In some cases, a module may be designed to be subdivided into sections, each of which may be jointly or separately controlled and/or monitored, for example, under different temperatures, relative humidities, pressures, gas concentrations, etc. Additionally, in some cases, more than one module may be placed or stacked together, for example, as is illustrated by modules 31, 32, and 33 in FIG. 3. The exact configuration and placement of modules will be a function of a particular application, the size and/or type of module, the available space, etc.

A module may be designed to be detachable from a base in some cases, for example, in embodiments where more than one module is brought to the device, for example, during operation of the device. As an example, a module that contains empty or unused chambers or other substrates for use during operation of the device may be brought into the device (e.g., replacing another module containing chambers or substrates), thereby allowing extended or continuous operation. As another example, a module that contains chambers filled with a product (e.g., after a reaction has been completed) may be removed from the device for further post-processing, and optionally replaced with an empty module ready to receive chambers. In some cases, the module may include mechanisms, such as wheels or casters, configured to be able to facilitate its movement to and from the device. In certain embodiments, the module may include a mechanism, such as a lock, that can secure the module to the device.

In certain cases, a module may be designed to have an opening able to facilitate and/or restrict the addition or removal of one or more chambers (or other substrates) from the module. For example, in one set of embodiments the module may include a port that is only large enough to readily admit one or a small number of chambers at a time, for example, as illustrated by access port 55 in FIG. 5. A smaller access port may be advantageous, for example, in cases where control of the internal environment of the module is desired. For instance, the module may be enclosed to keep out dust and/or other external contaminants, such as airborne contaminants, from entering the module, with access through the opening. A smaller access port may reduce the exchange of gases and/or changes in environmental conditions between the module and the external environment around the module, for instance, while chambers or other substrates are being added to or removed from the module. The access port may be designed in some embodiments such that an operator, such as a user or an external mechanism (e.g., a handling apparatus), is able to add or remove one or a small number of chambers from the module. In certain embodiments, though, the access port may be a simple opening within the module. In one set of embodiments, the access port includes a minimum cross-sectional dimension that is no greater than 4 times the minimum dimension of a chamber or other substrate introduced through the access port. Alternatively, the minimum dimension can be no more than 3 times, 2 times, or 1.5 times the minimum dimension of a chamber or substrate introduced through the access port. As used herein, the “minimum dimension” is the distance between two parallel, imaginary planes, positioned as close to each other as possible, between which the entire substrate can reside. Defined another way in connection with a generally rectangular solid shape, having a length, width, and height or thickness, the height or thickness of the shape defines the minimum dimension and is less than each of the length and width.

The access port may be controllable in some cases, for example, between an open state and a closed state. For example, the access port may be normally closed, but be openable as needed by an operator (e.g., a user or a handling system), for example, at certain preset times (such as with a door). In some cases, the access port to the module may include an airlock, e.g., an apparatus having more than one door that has to be opened and closed in series in order for internal access to occur. In certain instances, the access port may be controlled through the use of self-sealing materials (i.e., a material that will not allow a liquid or a gas to readily pass therethrough without the application of an external driving force, but will admit the insertion of a needle or other mechanical device able to penetrate the material). Examples of self-sealing systems include plastic flaps that cover the access port when the access port is not in use, or a material that blocks the access port and can be mechanically penetrated as desired.

In certain embodiments, one or more modules and/or handling apparatuses may be enclosed within a housing, for example, to maintain cleanliness and/or sterility of the interior of the module(s) and/or any chambers (or other substrates) contained therein. As an example, in FIG. 4D, housing 40 encloses modules 30, 31, 32, 33, 34, 35, 36, 37 and handling apparatus 20. The housing is able to maintain a controlled environment therein in some cases. For instance, in one embodiment, the housing is able to maintain a substantially sterile environment and/or a substantially particle- and/or dust-free environment therein. Thus, the housing may be able to keep external bacteria and/or other airborne contaminants within the housing to an acceptably low or negligible level in some cases, e.g., at a level unable to cause significant contamination or alteration of any reactions occurring within the chamber or other substrate. For example, if a device of the invention is able to manipulate one or more chambers comprising cells, then the housing containing the device (or portion thereof) may be able to prevent the cells within the chambers from being contaminated by airborne bacteria, viruses, etc., or the housing may be able to create an environment therein suitable for growing and/or maintaining the cells within the chambers. In various embodiments of the invention, the housing can also part of an incubator, a refrigerator, or a freezer.

Of course, in other embodiments, the device of the invention is not contained within a housing, for example, in embodiments where non-sterile operations or other operations not affected by external conditions are being performed (e.g., as in certain chemical reactions), or in embodiments where sterility and/or cleanliness is controlled through some other means, such as through chambers or modules that are enclosed or resistant to contamination. Thus, in one set of embodiments, the device may be exposed to ambient environmental conditions (e.g., ambient temperature, ambient air, ambient humidity, etc.), or conditions containing an appreciable particle density, for example, containing at least about 10,000 particles/m³, at least about 30,000 particles/m³, at least about 100,000 particles/m³, at least about 300,000 particles/m³, at least about 1,000,000 particles/m³, at least about 3,000,000 particles/m³, at least about 10,000,000 particles/m³, at least about 40,000,000 particles/m³, or at least about 100,000,000 particles/m³.

Other non-limiting examples of modules that may be provided in certain embodiments to manipulate a chamber or other substrate include certain commercially available devices, for example, Freedom EVO, Genesis RSP, or Genesis NPS, each from Tecan (Maennedorf, Switzerland). In certain embodiments, one or more modules and/or handling apparatuses described above may be controlled by an operator (e.g., a mechanical or automated system, or a human user). A device according to certain embodiments may be configured so that a human user may control operation of the modules and/or handling apparatuses, for example, using a user interface (such as a control panel), or a computer, as further described below. In other embodiments, however, a device may be programmable and/or automated, for example, such that the device is able to respond to a certain condition, or reach a certain level of productivity. Of course, in some embodiments, a device is both user-controlled and automated, for example, in cases where a user is able to override or alter an automated program.

In one set of embodiments, a device is configured so that a user is able to operate and/or control the device, for example, by programming the device to respond to, manipulate, or determine a certain cell type, a certain chamber or chamber design, a certain environmental condition, etc. The user interface may be configured to allow a user to input one or more parameters affecting a particular experiment, and/or inspect and/or monitor any aspect of an experiment or process being performed by the device. For example, the user interface may be configured to allow programming or manual control of any or all of the modules and/or handling apparatuses, data display, data analysis or determination, data storage, data handling, etc. In some cases, the data may be determined and/or manipulated in real-time.

In some cases, the user interface may also include a data management system. The data management system may be configured to allow, for example, searching of data generated by the device. The data generated by the device may include, but is not limited to, the initial state of one or more chambers or other substrates, concentrations, the type of cell line (if any), cell density, type of media, the pH, temperatures or pressures within the device or within the chambers or substrates, the pH or other set points, atmospheric or other environmental conditions within the device or within the chambers or substrates, identification of the chambers or other substrates within the device (e.g., using a bar code), data acquired from sensors or assay modules, images such as optical or fluorescent images, time data (e.g., time stamps), etc. The data may also be exported to other platforms for further analysis in some cases.

The user interface, in one set of embodiments, may be configured to allow a large number of parameters to be analyzed, for example, as in factorial analysis. The interface may be configured so that parameters leading to an optimized solution (e.g., maximizing a reaction rate, chemical yield, enantioselectivity, or, in the case of cells, cell growth, cell yield, cell division, production of one or more desired compounds, etc.) may be chosen for further study, and/or for further scale-up and/or “numbering-up,” as further described below.

As an example, an experiment may be performed where tens, hundreds or even thousands of parameters are varied systematically, e.g., using a factorial design algorithm. As one example, where cells are present in the device, factors that could be determined include, but are not limited to, temperatures, pressures, initial pHs, pHs during a reaction, media compositions (e.g., glutamine, sugars, carbohydrates, hormones, vitamins, serum, sources of nitrogen and/or carbon, etc.), flowrates, dissolved gas concentrations (e.g., O₂, CO₂, N₂, etc.), cell types, cell densities, cell cycle positions, cell dimensions, substrates, shear rates, gas concentrations, relative humidities, cell synthesis or production rates, cell replication rates, etc. Optimized conditions could be selected, e.g., for further study, or for scaling or “numbering.” In certain embodiments, it is possible to simultaneously process more than one chamber (or other substrate) using the systems and methods described herein. For example, an embodiment of the invention may configured to be able to process least five chambers or substrates simultaneously, or in other cases at least about 6, at least about 7, about 10, about 50, about 100, about 500, about 1,000, about 5,000, about 10,000, about 50,000, or even about 100,000 or more chambers or substrates simultaneously. In some cases, the number of chambers or substrates provided may be selected so as to produce a certain quantity of a species or product, or so as to be able to process a certain amount of reactant at a certain rate. Thus, certain embodiments of the invention are amenable to scalability and parallelization.

With regard to throughput, in certain embodiments, inventive devices may be configured so that multiple chambers (or other substrates) may be simultaneously processed to generate larger capacities. Additionally, in certain embodiments, an advantage may be obtained by maintaining production capacity at the small scale of reactions typically performed in the laboratory, with scale-up via parallelization. Scale-up, in the context of a chemical or biological process, may thus occur by means of adding more chambers (or other substrates) to the system (“numbering up”), rather than only, or in addition to, increasing the size and/or volume of the chamber(s). Certain embodiments of the invention may be used for the production of certain compounds, such as fine chemicals or pharmaceutical agents, on a large scale, e.g., by using chemical synthetic routes and/or biological or cellular processes.

As another example, a device of certain embodiments of the invention may be configured to be able to “reformat” a sample, e.g., from one chip or substrate to another chip or substrate, or from one region of a chip or substrate to another region on the same chip or substrate. As used herein, “reformat,” in reference to chips or substrates, refers to the redistribution of a first number of samples from the first chip or substrate, to a second number of samples within the second chip or substrate, where the second number is different from the first number. For example, in a reformatting operation, the system may reformat 1 sample into 6 samples, 6 samples into 24 samples, 1 sample into 96 samples, 1 sample into 384 samples, 6 samples into 96 samples, 96 samples into 6 samples, 96 samples into 24 samples, 96 samples into 384 samples, etc. The actual reformatting parameters will depend on the particular application, as would be understood by those of ordinary skill in the art.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one act, the order of the acts of the method is not necessarily limited to the order in which the acts of the method are recited.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A system, comprising:
an apparatus constructed and arranged to secure a biological substrate, wherein the apparatus is able, independently, to rotate the biological substrate about an axis, and translationally move the biological substrate in at least one of a direction substantially perpendicular to the axis and a direction substantially parallel to the axis.

2. A system as in claim 1, wherein the apparatus is constructed and arranged to secure a chamber containing the biological substrate.

3. A system as in claim 2, wherein the chamber is a cell culture chamber.

4. A system as in claim 1, wherein the apparatus is cylindrically coordinated.

5. A system as in claim 1, wherein the apparatus is articulated.

6. A system as in claim 1, wherein the apparatus comprises a linear translation stage.

7. A system as in claim 1, wherein the apparatus is configured to be able to, independently, translationally move the biological substrate in a direction substantially perpendicular to the axis, and translationally move the biological substrate in a direction substantially parallel to the axis.

8. A system as in claim 1, further including a sensing module.

9. A system as in claim 1, further including an incubator.

10. A system as in claim 1, further comprising a sterilization module constructed and arranged to sterilize at least a portion of at least one of the apparatus and the biological substrate.

11. A system as in claim 1, further including an identification module.

12. A system as in claim 1, further including a data transfer module.

13. A system as in claim 1, further including an imaging module.

14. A system as in claim 1, further including a fluid transfer module.

15. A system as in claim 14, wherein the fluid transfer module is configured to be able to transfer less than about 1 ml of fluid with respect to the biological substrate.

16. A system as in claim 15, wherein the fluid transfer module is configured to be able to transfer less than about 100 microliters of fluid with respect to the biological substrate.

17. A system as in claim 16, wherein the fluid transfer module is configured to be able to transfer less than about 10 microliters of fluid with respect to the biological substrate.

18. A system as in claim 17, wherein the fluid transfer module is configured to be able to transfer less than about 1 microliter of fluid with respect to the biological substrate.

19. A system as in claim 18, wherein the fluid transfer module is configured to be able to transfer less than about 300 nl of fluid with respect to the biological substrate.

20. A system as in claim 19, wherein the fluid transfer module is configured to be able to transfer less than about 100 nl of fluid with respect to the biological substrate.

21. A system as in claim 20, wherein the fluid transfer module is configured to be able to transfer less than about 30 nl of fluid with respect to the biological substrate.

22. A system as in claim 21, wherein the fluid transfer module is configured to be able to transfer less than about 10 nl of fluid with respect to the biological substrate.

23. A system as in claim 22, wherein the fluid transfer module is configured to be able to transfer less than about 3 nl of fluid with respect to the biological substrate.

24. A system as in claim 23, wherein the fluid transfer module is configured to be able to transfer less than about 1 nl of fluid with respect to the biological substrate.

25. A system as in claim 14, wherein the fluid transfer module is configured to be able to add a substance to the biological substrate.

26. A system as in claim 14, wherein the fluid transfer module is configured to be able to remove a substance from the biological substrate.

27. A system as in claim 14, wherein the fluid transfer module comprises a pump configured to be able to pump the substance in at least two directions.

28. A system as in claim 14, wherein the fluid transfer module comprises a needle manifold.

29. A system as in claim 14, wherein the fluid transfer module comprises a reservoir.

30. A system as in claim 1, further comprising a module configured to be able to spectroscopically determine a species at at least a portion of the biological substrate.

31. A system as in claim 1, further comprising a module configured to be able to determine pH of at least a portion of the biological substrate.

32. A system as in claim 1, further comprising a waveguide positioned to be in optical communication with at least a portion of the biological substrate.

33. A system as in claim 1, further comprising a module configured to be able to contain the biological substrate.

34. A method, comprising acts of:
directing an apparatus to remove a biological substrate from a first module configured to be able to perform a manipulation on the biological substrate;
rotating at least a portion of the substrate about an axis; and
directing the apparatus to position the biological substrate in a second module configured to be able to perform a manipulation on the biological substrate.

35. A method as in claim 34, wherein at least one of the first module and the second module is an incubator.

36. A method as in claim 34, further comprising determining a characteristic of the biological substrate.

37. A method as in claim 36, wherein the characteristic is protein concentration.

38. A method as in claim 36, wherein the characteristic is a concentration of a small molecule.

39. A method as in claim 34, wherein the biological substrate contains at least one cell.

40. A method as in claim 39, further comprising determining a characteristic of the at least one cell.

41. A method as in claim 40, wherein the characteristic is cell density.

42. A method as in claim 40, wherein the characteristic is cell viability.

43. A method as in claim 34, further comprising an act of:
directing a second apparatus to remove the biological substrate from the second module.

44. A method, comprising an act of:
subjecting at least one biological substrate to a plurality of different environmental conditions using an apparatus constructed and arranged to secure a substrate, wherein the apparatus is configured to be able to independently rotate the substrate about an axis.

45. A method of selecting an environmental condition, comprising acts of:
subjecting at least two predetermined reaction sites, each having a volume of less than about 1 ml, each to a different environmental condition;
selecting an environmental condition having a desired effect on a species within one of the at least two predetermined reaction sites; and
applying the selected environmental condition in a reactor containing cells.

46. A method as in claim 45, wherein at least one of the plurality of predetermined reaction sites contains cells.

47. A method as in claim 45, wherein the at least two predetermined reaction sites contains more than one cell type.

48. A method as in claim 45, wherein the reactor containing cells has a volume of greater than about 1 ml.

49. A method as in claim 45, wherein the characteristic is cell density.

50. A method as in claim 45, wherein the characteristic is cell yield.

51. A method as in claim 45, wherein the characteristic is cell viability.
